(19) United States
(12) Patent Application Publication
(10) Pub. No.: US 2005/0266086 A1
(43) Pub. Date: Dec. 1, 2005

(54) Intrauterine applications of materials formed in situ

(76) Inventor: Amarpreet S. Sawhney, Lexington, MA (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/858,516

(22) Filed: Jun. 1, 2004

(51) Int. Cl. A61K 31/765; A61K 9/14

(52) U.S. Cl. 424/486; 424/78.37

(57) ABSTRACT

Certain embodiments herein are directed to method of preventing adhesions in a uterus by introducing a flowable material into a uterus to tamponade a surface of the uterus. Such a material may be a hydrogel. The hydrogel may be formed in situ from at least one precursor, for example, a hydrophilic polymer with functional groups for forming covalent bonds.

### FIELD OF USE

Aspects of the invention relate to materials delivered to a uterus, including hydrogels formed in situ in the uterus from at least one precursor.

### BACKGROUND

The unwanted adherence of tissues to each other following medical intervention, an event termed an adhesion, is a complication that can lead to painful and debilitating medical problems. The presence of adhesions within the uterine cavity can lead to infertility. Surgical resection of these adhesion has a high rate of adhesion re-formation due to the close proximity of the uterine walls. Conventional technologies for preventing intrauterine adhesions have limited effectiveness.

### SUMMARY OF THE INVENTION

Materials and methods for preventing intrauterine adhesions are presented herein. These technologies may also be used to stop unwanted bleeding post-resection and to provide mechanical support for uterine tissues. Materials may be introduced into the uterus to contact tissues of the uterus to reduce or prevent contact between the tissues, or portions of the tissues. Flowable components may be used so as to ease the introduction and formation of the materials. For example, at least one precursor may be introduced into the uterus to form a material in the uterus after its introduction. Examples of precursors include polymerizable, crosslinkable, and thermosetting polymers that form a material, e.g., a hydrogel, inside the uterus.

Some embodiments relate to a method of preventing adhesion in a uterus, the method comprising introducing a flowable material into a uterus to tamponade a surface of the uterus. The tamponade can be effective to reduce bleeding from resected tissues. The material may be, e.g., a hydrogel and may function as a stent or a splint. Some embodiments relate to a method of preventing adhesion in a uterus by crosslinking at least one precursor to form a hydrogel in the uterus, e.g., to tamponade a surface of the uterus or to prevent the collapse and adherence of the uterine walls to each other.

### BRIEF DESCRIPTION OF THE FIGURES

FIGS. 1A and 1B are, respectively, a side view and cross-sectional view, taken along view line 1B--1B, of a delivery system for injecting two precursors to into a body lumen;

FIG. 2 illustrates a method of using the apparatus of FIG. 1 to treat a uterus;

FIG. 3 depicts a cross-sectional view of an alternative embodiment of a delivery system for a composition as described herein;

FIG. 4 depicts a cross-sectional view of an alternative embodiment of a delivery system for a composition as described herein;

FIG. 5 depicts electrophilic, water soluble, and biodegradable precursors,

FIG. 6 depicts nucleophilic, water soluble and biodegradable precursors,

FIG. 7 depicts water soluble and biodegradable precursors wherein either the biodegradable linkages or the functional groups are selected so as to make the precursor water soluble,

FIG. 8 depicts water soluble precursors which are not biodegradable,

FIG. 9 depicts water soluble precursors which are not biodegradable,

FIG. 10 depicts the preparation of an electrophilic precursor using carbodiimide (“CDI”) activation chemistry, its crosslinking reaction with a nucleophilic water soluble functional polymer to form a biocompatible crosslinked polymer product, and the hydrolysis of that biocompatible crosslinked polymer to yield water soluble fragments,

FIG. 11 depicts the use of sulfonyl chloride activation chemistry to prepare a precursor,

FIG. 12 depicts the preparation of an electrophilic water soluble precursor using N-hydroxysuccinimide (“NHS”) activation chemistry, its crosslinking reaction with a nucleophilic water soluble precursor to form a biocompatible crosslinked polymer product, and the hydrolysis of that biocompatible crosslinked polymer to yield water soluble fragments,

FIG. 13 depicts preferred NHS esters,

FIG. 14 shows the N-hydroxysulfosuccinimide (“SNHS”) activation of a tetrafunctional sugar-based water soluble synthetic precursor and its crosslinking reaction with 4-arm amine terminated polyethylene glycol precursor to form a biocompatible crosslinked polymer product, and the hydrolysis of that biocompatible crosslinked polymer to yield water soluble fragments, and

FIGS. 15-18 show ultrasound images as described in the Example.

### DETAILED DESCRIPTION OF PREFERRED EMBODIMENTS

Intrauterine Adhesions

Adhesions are fibrous bands of tissue that may form as a result of trauma to internal surfaces, including trauma to the endometrial lining of the uterine cavity. To date, some attention has been focused on intraperitoneal adhesion formation¹⁻⁴. However, adhesions can also form within the uterine cavities. Adhesions may lead to partial or total blockage of the uterine cavity. Such a loss may result in abnormal bleeding, infertility, and recurrent pregnancy loss³. Indeed, the incidence of intrauterine adhesion formation following such procedures as dilatation and curettage following late stage abortions can be as high as 30 to 50%^{4,5}. And adhesion reformation rates following hysteroscopic adhesiolysis, may reach as high as 60%³. The real incidence of adhesions after operative hysteroscopy is unknown but it is almost clear that any factor leading to destruction of the endometrium may engender adhesions of the myometrium at the opposite walls of the uterus¹². In these conditions while treating the primary cause of subfertility, one risks creating adhesions, which present a more insidious risk to fertility. Many factors can be presumed to affect the differences in the incidence of intrauterine adhesion formation reported. The skill of the surgeon, technique and instrumentation used for resection, patient predilection for adhesion formation, and missed intrauterine infections may be some important factors in this regard. However, there has been a notable lack of therapies specifically directed to adhesion prophylaxis following hysteroscopic surgery.

Even thin, relatively avascular adhesions may impair fertility⁶. The association between presence of adhesions and infertility is so great that inducement of adhesions has been proposed as an effective method of contraception⁷. Furthermore, there is evidence that the severity of adhesions may be progressive: mild, filmy adhesions may advance to fibromuscular adhesions still composed of endometrium, ultimately developing into dense connective tissue lacking endometrium altogether^{4,8}. Most investigators agree that the incidence of intrauterine adhesion formation has risen with increased elective interventions⁹. However, restoration of normal menstruation and improved fertility rates can be achieved in patients treated with hysteroscopic adhesiolysis, although the fertility outcome tends to parallel the severity stage of the adhesions already formed².

Prevention of intrauterine adhesions may be useful when, e.g., the patient is infertile or has had one or more abortions and wishes to conceive. Several conditions that may impair fertility or lead to recurrent abortions are the presence of uterine septa, endometrial polyps, submucous fibroids, or intrauterine synechiae¹¹ that may require hysteroscopic resection. The trauma resulting from resection or aggressive D&C after abortion on the walls of the uterine cavity can provoke the development of intrauterine adhesions.

The proposed mechanism for the progressive nature of the disease reflects a cycle: “younger” less dense adhesions limit uterine muscular activity, reducing perfusion of estrogen to the endometrium, and eventually resulting in the final transformation from fibrous connective bands to myometrium devoid of endometrial elements^{2,4,8,10}. Once the more severe form of adhesions has formed, endometrial malfunction is more pronounced and appears to carry a worse prognosis^{2,4,8,10}. The gravid uterus is particularly predisposed to adhesion formation, which means that the population of patients who have suffered an interrupted pregnancy are at the highest risk of continued, increasingly severe problems of infertility^{9,11}. Curettage during the two to four weeks postpartum presents the highest risk of inducing adhesion formation, because the traumatized endometrium is particularly vulnerable^{4,9}. Therefore, if a method is identified which can prevent the formation of adhesions of any severity, the cycle may be prevented, and fertility improved in these patients.

The Example, below, described the delivery of precursors to the uterus to form hydrogels therein. The Example used a material referred to as SPRAYGEL, obtained from Confluent Surgical, Inc, Boston, Mass. Earlier studies of SPRAYGEL have demonstrated that is useful for prevention intraperitoneal adhesion formation^{5,6}. SPRAYGEL includes two liquids (one clear and one blue) that each contain chemically distinct precursors which, when mixed together, rapidly cross-link to form a biocompatible absorbable hydrogel in situ. Additional details for SPRAYGEL are provided in U.S. patent Ser. No. 10/010,715, filed Nov. 9, 2001, hereby incorporated by reference herein. The in situ polymerization occurs very rapidly (within seconds) with no heat evolved and no external energy source required (i.e., light source). Upon applying the two liquids through the “Y” blending connector, the liquids mix and cross-link to form a thin, flexible, tissue adherent barrier. The mixed liquids are delivered to the target site via an 8Fr applicator. Within about one to about two weeks of application, the adhesion barrier undergoes hydrolysis, and is absorbed into the circulatory system, and is excreted through the kidneys.

Significantly, the hydrogel described in the Example acted as a stent and a tamponade. A stent is a device that provides support to a structure. Thus, a tissue stent supports a tissue. In the case of an intravascular stent, a lumen for passage of blood therethrough is provided. A tamponade is a device or material that applies a compressive pressure against a tissue with enough force to reduce bleeding. In the case of a hydrogel, a tamponading force may be the result of an initial pressure created immediately after its introduction, or the result of subsequent swelling of the hydrogel or tissue. In either case, the hydrogel may be placed so that it can effectively exert such a force to achieve a tamponading effect.

Hydrogels

Hydrogels are materials that absorb solvents (such as water), undergo swelling without discernible dissolution, and maintain three-dimensional networks capable of reversible deformation. See, e.g., Park, et al., Biodegradable Hydrogels for Drug Delivery, Technomic Pub. Co., Lancaster, Pa. (1993).

Covalently crosslinked networks of hydrophilic polymers, including water-soluble polymers are traditionally denoted as hydrogels (or aquagels) in the hydrated state. Hydrogels have been prepared based on crosslinked polymeric chains of methoxypoly(ethylene glycol) monomethacrylate having variable lengths of the polyoxyethylene side chains, and their interaction with blood components has been studied (Nagaoka et al., in Polymers as Biomaterial (Shalaby et al., Eds.) Plenum Press, 1983, p. 381). A number of aqueous hydrogels have been used in various biomedical applications, such as, for example, soft contact lenses, wound management, and drug delivery.

Non-degradable hydrogels made from poly(vinyl pyrrolidone) and methacrylate have been fashioned into fallopian tubal occluding devices that swell and occlude the lumen of the tube. See, Brundin, “Hydrogel Tubal Blocking Device: P-Block”, in Female Transcervical Sterilization, (Zatuchini et al., Eds.) Harper Row, Philadelphia (1982). Because such hydrogels undergo a relatively small amount of swelling and are not absorbable, so that the sterilization is not reversible, the devices described in the foregoing reference have found limited utility.

Hydrogels may be absorbable or non absorbable in nature. They can be formed from physical or chemical crosslinking or both. Hydrogels that for the invention may be delivered through substantially non-invasive means, such as a catheter. Thus, the hydrogel itself may either be thixotrophic or may be formed in-situ after delivery. The hydrogel may begin as one or more liquid precursor solutions that can form into a gel upon activation. The activation may be provided by either mixing with another component or by encountering a condition within the body cavity, or the uterus, that enables the formation of the hydrogel, for example, by heat activated initiation of a free radical generating species, that can polymerize a free radically polymerizable macromeric species.

Certain embodiments are directed to methods and apparatus for intraluminally delivering two or more crosslinkable solutions to form hydrogel implants in situ. Included herein are dual- and multi-component hydrogel systems for such use and delivery systems for depositing such hydrogel systems. Some embodiments involve forming a material from a precursor. A precursor is a substance that becomes integrated into structure of the material that it forms. A functionalized polymer, a monomer, or a macromer used to form a gel or hydrogel would typically be a precursor, but an activation agent such as initiator would typically not be a precursor.

Crosslinkable solutions for use include those that may be used to form implants in lumens or voids, and may form physical crosslinks, chemical crosslinks, or both. Physical crosslinks may result from complexation, hydrogen bonding, desolvation, Van der Waals interactions, ionic bonding, etc., and may be initiated by mixing two components that are physically separated until combined in situ, or as a consequence of a prevalent condition in the physiological environment, such as temperature, pH, ionic strength, etc. Chemical crosslinking may be accomplished by any of a number of mechanisms, including free radical polymerization, condensation polymerization, anionic or cationic polymerization, step growth polymerization, etc. Where two solutions are employed, each solution preferably contains one component of a co-initiating system and crosslink on contact. The solutions are separately stored and mix when delivered into a tissue lumen.

Hydrogels may be crosslinked spontaneously from at least one precursor without requiring the use of a separate energy source. Such systems allow for control of the crosslinking process, e.g., because a large viscosity increase of materials flowing through a delivery device does not occur until after the device is in place. In the case of a dual-component system, mixing of the two solutions takes place so that the solutions are fluid while passing through the device. If desired, one or both crosslinkable precursor solutions may contain contrast agents or other means for visualizing the hydrogel implant. Alternatively, a colored compound may be produced as a byproduct of the reactive process. The crosslinkable solutions may contain a bioactive drug or other therapeutic compound that is entrapped in the resulting implant, so that the hydrogel implant serves a drug delivery function.

Properties of the hydrogel system, other than crosslinkability, preferably should be selected according to the intended application. For example, if the hydrogel implant is to be used to temporarily occlude a reproductive organ, such as the uterine cavity, it is preferable that the hydrogel system undergo significant swelling and be biodegradable. Alternatively, the hydrogel may have thrombotic properties, or its components may react with blood or other body fluids to form a coagulum.

Other applications may require different characteristics of the hydrogel system. There is extensive literature describing the formulation of crosslinkable materials for particular medical applications, which formulae may be readily adapted for use herein with little experimentation. More generally, the materials should be selected on the basis of exhibited biocompatibility and lack of toxicity. Also, the hydrogel solutions should not contain harmful or toxic solvents.

Additionally, the hydrogel system solutions maybe prepared without harmful or toxic solvents. Preferably, the solutions are substantially soluble in water to allow application in a physiologically-compatible solution, such as buffered isotonic saline. Water-soluble coatings may form thin films, but more preferably form three-dimensional gels of controlled thickness. A coating may be biodegradable, so that it does not have to be retrieved from the body. Biodegradability, as used herein, refers to the predictable disintegration of the coating into molecules small enough to be metabolized or excreted under normal physiological conditions. Biodegradability may occur by, e.g., hydrolysis, enzymatic action, or cell-mediated destruction.

Polymers for Physical Crosslinking

Physical crosslinking may be intramolecular or intermolecular or in some cases, both. For example, hydrogels can be formed by the ionic interaction of divalent cationic metal ions (such as Ca+2 and Mg+2) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. These crosslinks may be easily reversed by exposure to species that chelate the crosslinking metal ions, for example, ethylene diamine tetraacetic acid. Multifunctional cationic polymers, such as poly(l-lysine), poly(allylamine), poly(ethyleneimine), poly(guanidine), poly(vinyl amine), which contain a plurality of amine functionalities along the backbone, may be used to further induce ionic crosslinks.

Hydrophobic interactions may induce physical entanglement, especially in polymers, that induces increases in viscosity, precipitation, or gelation of polymeric solutions. For example, poly(oxyethylene)-poly(oxypropylene) block copolymers, available under the trade name of PLURONIC®, BASF Corporation, Mount Olive, N.J., are well known to exhibit a thermoreversible behavior in solution. Thus, an aqueous solution of 30% PLURONIC® F-127 is a relatively low viscosity liquid at 4° C. and forms a pasty gel at physiological temperatures due to hydrophobic interactions. Other block and graft copolymers of water soluble and insoluble polymers exhibit similar effects, for example, copolymers of poly(oxyethylene) with poly(styrene), poly(caprolactone), poly(butadiene) etc.

Techniques to tailor the transition temperature, i.e. the temperature at which an aqueous solution transitions to a gel due to physical linking may advantageously be used to make hydrogels. For example, the transition temperature may be lowered by increasing the degree of polymerization of the hydrophobic grafted chain or block relative to the hydrophilic block. Increase in the overall polymeric molecular weight, while keeping the hydrophilic: lipophilic ratio unchanged also leads to a lower gel transition temperature, because the polymeric chains entangle more effectively. Gels likewise may be obtained at lower relative concentrations compared to polymers with lower molecular weights.

Solutions of other synthetic polymers such as poly(N-alkylacrylamides) also form hydrogels that exhibit thermoreversible behavior and exhibit weak physical crosslinks on warming. During deposition of thermoreversible solutions, the solutions may cooled so that, upon contact with tissue target at physiological temperatures, viscosity increases as a result of the formation of physical crosslinks. Similarly, pH responsive polymers that have a low viscosity at acidic or basic pH may be employed, and exhibit an increase in viscosity upon reaching neutral pH, for example, due to decreased solubility.

For example, polyanionic polymers such as poly(acrylic acid) or poly(methacrylic acid) possess a low viscosity at acidic pHs that increases as the polymers become more solvated at higher pHs. The solubility and gelation of such polymers further may be controlled by interaction with other water soluble polymers that complex with the polyanionic polymers. For example, it is well known that poly(ethylene oxides) of molecular weight over 2,000 dissolve to form clear solutions in water. When these solutions are mixed with similar clear solutions of poly(methacrylic acid) or poly(acrylic acid), however, thickening, gelation, or precipitation occurs depending on the particular pH and conditions used (for example see Smith et al., “Association reactions for poly(alkylene oxides) and poly(carboxylic acids),” Ind. Eng. Chem., 51:1361 (1959). Thus, a two component aqueous solution system may be selected so that the first component (among other components) consists of poly(acrylic acid) or poly(methacrylic acid) at an elevated pH of around 8-9 and the other component consists of (among other components) a solution of poly(ethylene glycol) at an acidic pH, such that the two solutions on being combined in situ result in an immediate increase in viscosity due to physical crosslinking.

Physical gelation also may be obtained in several naturally existing polymers too. For example gelatin, which is a hydrolyzed form of collagen, one of the most common physiologically occurring polymers, gels by forming physical crosslinks when cooled from an elevated temperature. Other natural polymers, such as glycosaminoglycans, e.g., hyaluronic acid, contain both anionic and cationic functional groups along each polymeric chain. This allows the formation of both intramolecular as well as intermolecular ionic crosslinks, and is responsible for the thixotropic (or shear thinning) nature of hyaluronic acid. The crosslinks are temporarily disrupted during shear, leading to low apparent viscosities and flow, and reform on the removal of shear, thereby causing the gel to reform.

Macromers for Chemical Crosslinking

Water soluble polymerizable polymeric monomers having a functionality >1 (i.e., that form crosslinked networks on polymerization) and that form hydrogels may be referred to herein as macromers.

Several functional groups may be used to facilitate chemical crosslinking reactions. When these functional groups are self condensible, such as ethylenically unsaturated functional groups, the crosslinker alone is sufficient to result in the formation of a hydrogel, when polymerization is initiated with appropriate agents. Where two solutions are employed, each solution preferably contains one component of a co-initiating system and crosslink on contact. The solutions are stored in separate compartments of a delivery system, and mix either when deposited on or within the tissue.

An example of an initiating system suitable for use in the present invention is the combination of a peroxygen compound in one solution, and a reactive ion, such as a transition metal, in another. Other initiating systems such as thermally or photochemically initiated systems may also be used. Other means for crosslinking macromers to form tissue implants in situ also may be advantageously used, including macromers that contain groups that demonstrate activity towards functional groups such as amines, imines, thiols, carboxyls, isocyanates, urethanes, amides, thiocyanates, hydroxyls, etc., which may be naturally present in, on, or around tissue. Alternatively, such functional groups optionally may be provided in the lumen as part of the hydrogel system.

Preferred hydrogel systems are those biocompatible single or multi-component systems that spontaneously crosslink when the components are activated either by an initiating system or by mixing two components, but wherein the two or more components are individually stable. Such systems include, for example, contain macromers that are di or multifunctional amines in one component and di or multifunctional oxirane containing moieties in the other component. Other initiator systems, such as components of redox type initiators, also may be used. The mixing of the two or more solutions may result in either an addition or condensation polymerization that further leads to the formation of an implant. Free radical initiating systems that depend on thermal initiation or photoinitiation may also be used to trigger the polymerization of ethylenically unsaturated monomers or macromers to form hydrogels.

Monomers

Monomers capable of being crosslinked to form a biocompatible implant may be used. The monomers may be small molecules, such as acrylic acid or vinyl caprolactam, larger molecules containing polymerizable groups, such as acrylate-capped polyethylene glycol (PEG-diacrylate), or other polymers containing ethylenically-unsaturated groups, such as those of U.S. Pat. No. 4,938,763 to Dunn et al, U.S. Pat. Nos. 5,100,992 and 4,826,945 to Cohn et al, U.S. Pat. Nos. 4,741,872 and 5,160,745 to De Luca et al., or U.S. Pat. No. 5,410,016 to Hubbell et al.

Monomers may include the biodegradable, water-soluble macromers described in U.S. Pat. No. 5,410,016, hereby incorporated herein by reference. These monomers are characterized by having at least two polymerizable groups, separated by at least one degradable region. When polymerized in water, they form coherent gels that persist until eliminated by self-degradation. In the most preferred embodiment, the macromer is formed with a core of a polymer that is water soluble and biocompatible, such as the polyalkylene oxide polyethylene glycol, flanked by hydroxy acids such as lactic acid, having acrylate groups coupled thereto. Preferred monomers, in addition to being biodegradable, biocompatible, and non-toxic, also will be at least somewhat elastic after crosslinking or curing.

It has been determined that monomers with longer distances between crosslinks are generally softer, more compliant, and more elastic. Thus, in the polymers of U.S. Pat. No. 5,410,016, increased length of the water-soluble segment, such as polyethylene glycol, tends to enhance elasticity. Molecular weights in the range of 10,000 to 35,000 of polyethylene glycol are preferred for such applications, although ranges from 1,000 to 500,000 also are useful.

Initiating Systems

Metal ions may be used either as an oxidizer or a reductant in redox initiating systems. For example, ferrous ions may be used in combination with a peroxide or hydroperoxide to initiate polymerization, or as parts of a polymerization system. In this case, the ferrous ions serve as a reductant. In other previously known initiating systems, metal ions serve as an oxidant.

For example, the ceric ion (4+ valence state of cerium) interacts with various organic groups, including carboxylic acids and urethanes, to remove an electron to the metal ion, and leave an initiating radical behind on the organic group. In such a system, the metal ion acts as an oxidizer. Potentially suitable metal ions for either role are any of the transition metal ions, lanthanides and actinides, which have at least two readily accessible oxidation states.

Some metal ions have at least two states separated by only one difference in charge. Of these, the most commonly used are ferric/ferrous; cupric/cuprous; ceric/cerous; cobaltic/cobaltous; vanadate V vs. IV; permanganate; and manganic/manganous. Peroxygen containing compounds, such as peroxides and hydroperoxides, including hydrogen peroxide, t-butyl hydroperoxide, t-butyl peroxide, benzoyl peroxide, cumyl peroxide, etc. may be used. Characteristic of an organic peroxide is the presence of the peroxy group —O—O—. The reactivity of a given peroxide is decided by the chemical composition of the rest of the molecule.

The organic peroxide general formula is R₁—O—O—R₂ or R₁—O—O—R₃—O—O—R₂, where R₁ and R₂ can be hydrogen, ester, aryl, alkyl or acyl groups and R₃ can be aryl, alkyl, ester.

Thermal initiating systems may be used rather than the redox-type systems described hereinabove. Several commercially available low temperature free radical initiators, such as V-044, available from Wako Chemicals USA, Inc., Richmond, Va., may be used to initiate free radical crosslinking reactions at body temperatures to form hydrogel implants with the aforementioned monomers.

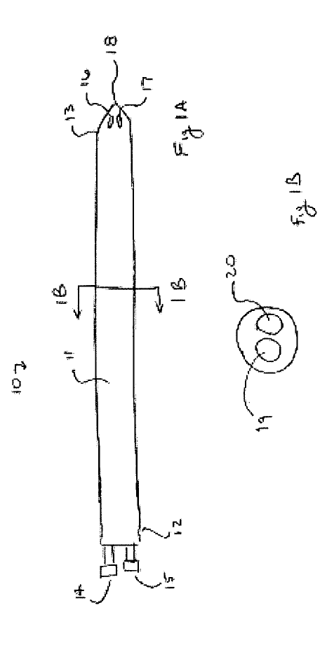

Delivery Systems for Forming Implants In Situ Referring to FIGS. 1A and 1B, an illustrative delivery system constructed in accordance with the principles of the present invention is described. Delivery system 10 comprises dual-lumen catheter 11 having proximal end 12 and distal end 13. Proximal end 12 includes inlet ports 14 and 15 coupled to respective outlet ports 16 and 17 disposed near tip 18 via separate lumens 19 and 20, respectively. In use, precursors are introduced into inlet orts 14 and 15 and allowed to mix after exiting from outlet ports 16 and 17.

Alternatively, a catheter may be configured to have only one lumen and two inlets via a Y connector. The two fluids are introduced in the Y connector and mix within the lumen of the catheter. They remain fluent until they exit the catheter and then rapidly polymerize. Each of the two fluids may comprise a precursor so that two precursors are mixed with the fluids. Mixing may initiate formation of a hydrogel, for example when the precursors each have functional groups that are reactive with functional groups on the other precursor. In general, precursors may be mixed before, during, or after delivery to the site, with formation of the hydrogel being completed at the site. The precursors may be flowable so as to flow into the site and conform to the shape of the site.

Injection of precursors may be continued without stopping so as to reduce plugging of the catheter or other delivery device due to formation of the hydrogel from the precursors. Alternatively, if a thermally polymerizing hydrogel is used, a single lumen catheter attached to a syringe containing the hydrogel forming precursor that contains the initiator already dissolved or dispersed in it may be used. After injecting and administration within the uterine cavity, the elevation of the hydrogel precursor to body temperature can trigger the activation of the initiation system and result in the formation of the hydrogel implant over time.

Delivery system 10 may be fabricated of any of a wide variety of materials that are sufficiently flexible and biocompatible. For example, polyethylenes, nylons, polyvinylchlorides, polyether block amides, polyurethanes, and other similar materials are suitable.

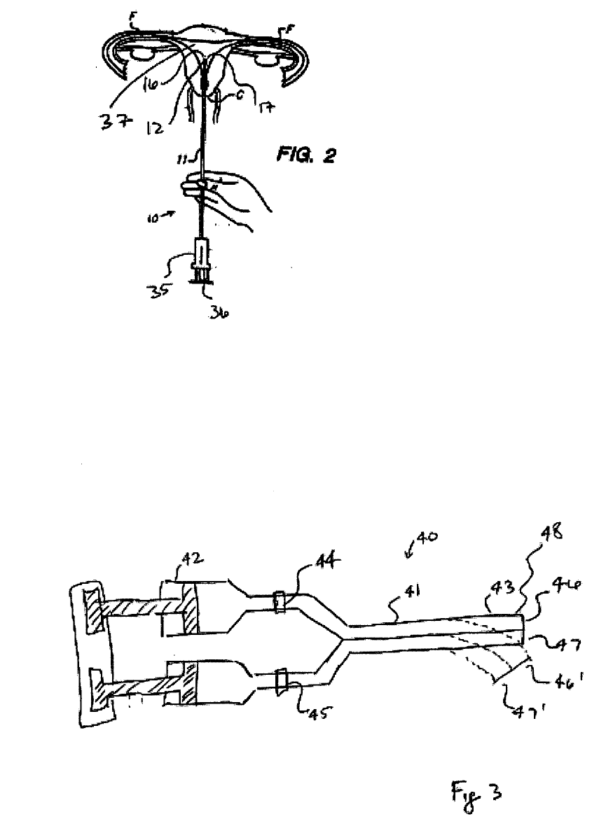

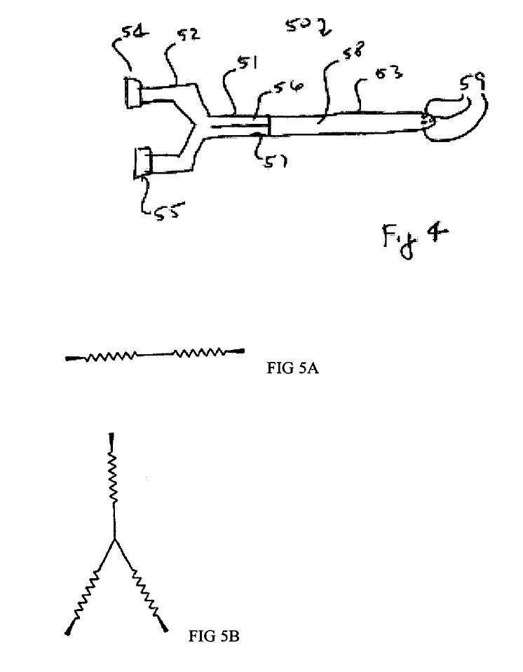

Delivery system 10 should be of a size appropriate to facilitate delivery, to have a minimum profile, and cause minimal trauma when inserted and advanced to a treatment site. In an embodiment suitable for forming hydrogel implants in the uterus, delivery system 10 preferably is no larger than about 4 mm to facilitate delivery through the cervix or a hysteroscope channel. Referring now to FIG. 2, a method of using delivery system 10 of FIG. 1 is described for delivering hydrogel-forming precursor materials within a uterine cavity. Proximal end 12 of delivery system 10 is coupled to dual syringe-type device 35 having actuator 36 that allows simultaneous injection of two precursor solutions to form a hydrogel. Actuator 36 is depressed so that solutions of precursor(s) are delivered through outlet ports 16 and 17 within the uterine cavity. The solutions are allowed to mix and crosslink, thus forming a hydrogel implant that occupies the uterine cavity.

While the deployment of the hydrogel is often done without imaging and in a blind fashion, it is possible to add imaging agents, such as microbubbles, to enable imaging under ultrasound or by adding a radioopacifying agent to enable imaging under X-ray guidance. If desired, the treatment space may be filled or flushed with a solution, such as an inert saline solution, to remove blood and other biological fluids from the treatment space prior to delivering the hydrogel. Delivery system 10 optionally may include an additional lumen to permit such flushing liquids to exit the treatment space. Alternatively, a non-inert solution, such as a solution containing a pharmaceutical agent, may be injected into the treatment space.

Imaging, as used herein, refers to methods of producing an image that involve use of a machine to make imaging agents visible. For example, an X-ray machine is used to make X-ray imaging agents visible, or an ultrasound machine is used to make microbubble ultrasound imaging agents visible. In contrast, a visualization agent is an agent that can be directly observed. A fluorescent agent that emits light in the visible spectrum would be a visualization agent while a fluorescent agent that emitted light outside the visible light range would be an imaging agent. Various embodiments are set forth herein that refer to a visualization agent; it would also be generally possible to add or substitute an imaging agent for those embodiments, provided that an imaging device can be used with that method.

As set forth in the Example, a dual syringe having two lumens for delivering two precursors separately to the site for the hydrogel may be used. The precursors mix after exiting the lumens and form a hydrogel. Alternatively, single or multiple precursors could be delivered.

Alternatively, a precursor or precursors and an activating agent could be delivered. An activating agent is an agent that initiates a precursor, or precursors, to form a gel such as a hydrogel. The activating agent could be, e.g., a polymerization initiator for use with polymerizable functional groups, or an ion for use with polymers that gel in response to exposure to an ion or any of several free radical generating thermal, chemical, or photochemical initiators known in the art. For example, photopolymerizable macromers delivered in combination with an initiator could be used, provided that a source of light for triggering the photopolymerization was also provided, e.g., as described in U.S. Pat. Nos. 6,387,977; 5,410,016; and 5,462,990, which are hereby incorporated herein by reference.

Referring now to FIG. 3, an alternative embodiment of a delivery system constructed in accordance with the principles of the present invention is described. Delivery system 40 comprises dual-lumen catheter 41 having proximal region 42 and flexible distal region 43. Proximal region 42 includes inlet ports 44 and 45 and outlet ports 46, 47 disposed on tip 48. One or more radio-opaque or ultrasound lucent marker bands (not shown) may be disposed in distal region 43 to assist in positioning delivery system 40 within a natural or induced body lumen under fluoroscopic or ultrasound guidance.

With respect to FIG. 4, a further alternative embodiment of a delivery system constructed in accordance with the principles of the present invention is described. Delivery system 50 comprises dual-lumen catheter 51 having proximal end 52 and distal end 53. Proximal end 52 includes inlet ports 54 and 55 coupled to lumens 56 and 57 that empty into lumen 58. Lumen 58 has exit ports 59. In use, a precursor is introduced into one of lumens 56 and 57 and a precursor or activating agent is introduced into the other of lumens 56 and 57. These are at least partially mixed in lumen 58 and are expelled from the device via at least one port 59. The precursor(s) form a hydrogel at the delivery site, e.g., by reaction of at least one precursor with another precursor or by reaction of at least one precursor in response to an activation agent. The activation agent may spontaneously activate the hydrogel formation without energy from a light source and/or without non-thermal energy and/or without an external energy source. Alternatively, the activation agent may require a light source, non-thermal energy, or an external energy source.

It is sometimes useful to provide color by adding a colored visualization agent to hydrogel precursors before crosslinking. The visualization agent may serve to help a user visualize the disposition of the hydrogel. For example, when filling a uterus, a visualization agent will help to distinguish the hydrogel from other fluids. Further, the hue of a colored hydrogel may provide information about the concentration of the precursors in the hydrogel or the degree of mixing of physiological fluids into the hydrogel. A dark color hydrogel may indicate a concentration of precursors that is high relative to a lighter hued hydrogel made from the same precursor solutions. The coloring agent may be present in a premixed amount that is already selected for the application. An embodiment of the invention uses biocompatible crosslinked polymers formed from the reaction of precursors having electrophilic functional group and nucleophilic functional groups. The precursors are preferably water soluble, non-toxic, and biologically acceptable.

In some embodiments, at least one of the precursors is a small molecule of about 1000 Da or less, and is referred to as a “small molecule crosslinker”. The small molecule crosslinker preferably has a solubility of at least 1 g/100 mL in an aqueous solution. A crosslinked molecule may be crosslinked via an ionic or covalent bond, a physical force, or other attraction. Preferably, at least one of the other precursors is a macromolecule, and is referred to as a “functional polymer”. The macromolecule, when reacted in combination with a small molecule crosslinker, is preferably at least five to fifty times greater in molecular weight than the small molecule crosslinker and is preferably less than about 60,000 Da. A more preferred range is a macromolecule that is seven to thirty times greater in molecular weight than the small molecule crosslinker and a most preferred range is about ten to twenty times difference in weight. Further, a macromolecular molecular weight of 5,000 to 50,000 is preferred, a molecular weight of 7,000 to 40,000 is more preferred and a molecular weight of 10,000 to 20,000 is most preferred. The term polymer, as used herein, means a molecule formed of at least three repeating groups. The term “reactive precursor species” means a polymer, functional polymer, macromolecule, small molecule, or small molecule crosslinker that can take part in a reaction to form a network of crosslinked molecules, e.g., a hydrogel.

An embodiment of the invention is a hydrogel for use on a patient's tissue that has water, a biocompatible visualization agent, and crosslinked hydrophilic polymers that form a hydrogel after delivery within the uterine cavity. The visualization agent reflects or emits light at a wavelength detectable to a human eye so that a user applying the hydrogel can observe the gel and also estimate its thickness.

Natural polymers, for example proteins or glycosaminoglycans, e.g., collagen, fibrinogen, albumin, and fibrin, may be crosslinked using reactive precursor species with electrophilic functional groups. Natural polymers are proteolytically degraded by proteases present in the body. Synthetic polymers and reactive precursor species are preferred, however, and may have electrophilic functional groups that are carbodiimidazole, sulfonyl chloride, chlorocarbonates, n-hydroxysuccinimidyl ester, succinimidyl ester or sulfasuccinimidyl esters. The term synthetic means a molecule that is not found in nature, e.g., polyethylene glycol. The nucleophilic functional groups may be, for example, amine, hydroxyl, carboxyl, and thiol. The polymers preferably have a polyalkylene glycol portion. More preferably they are polyethylene glycol based. The polymers preferably also have a hydrolytically biodegradable portion or linkage, for example an ester, carbonate, or an amide linkage. Several such linkages are well known in the art and originate from alpha-hydroxy acids, their cyclic dimmers, or other chemical species used to synthesize biodegradable articles, such as, glycolide, dl-lactide, l-lactide, caprolactone, dioxanone, trimethylene carbonate or a copolymer thereof. A preferred embodiment has reactive precursor species with two to ten nucleophilic functional groups each and reactive precursor species with two to ten electrophilic functional groups each. The hydrophilic species are preferably synthetic molecules.

Preferred biocompatible visualization agents are FD&C BLUE #1, FD&C BLUE #2, and methylene blue. These agents are preferably present in the final electrophilic-nucleophilic reactive precursor species mix at a concentration of more than 0.05 mg/ml and preferably in a concentration range of at least 0.1 to about 12 mg/ml, and more preferably in the range of 0.1 to 4.0 mg/ml, although greater concentrations may potentially be used, up to the limit of solubility of the visualization agent. These concentration ranges were found to give a color to the hydrogel that was desirable without interfering with crosslinking times (as measured by the time for the reactive precursor species to gel). The visualization agent may also be a fluorescent molecule. The visualization agent is preferably not covalently linked to the hydrogel.

An embodiment is a hydrogel that at least partially fills a uterus. An embodiment is a hydrogel that substantially fills a uterus. An embodiment is a hydrogel shaped like an interior of a uterus. An embodiment is a hydrogel that forms a coating on at least a portion of an intrauterine tissue. An embodiment is a hydrogel that substantially fills a uterus and has contact with substantially all of the tissues exposed inside the uterus. The introduction of fluent precursor(s) or precursor solutions into a uterus that form a hydrogel having a volume that is essentially equal to the volume of the fluent precursor(s) or precursor solutions will contact substantially all of the tissues exposed inside the uterus because a fluid will conform to the shape of the tissues. Nonetheless, it is appreciated by persons of ordinary skill in the art that even substantially complete contact may suffer from imperfections.

An embodiment is a method of use is to form a hydrogel on a tissue until the color of the hydrogel indicates that a predetermined volume of hydrogel has been deposited on the tissue or within the space. An embodiment is a method of introducing at least one precursor into a tissue space to form a hydrogel from the precursor(s). The precursor(s) may be associated with a visualization agent. The precursors are continually introduced into the space until the color of the materials that enter that space and flow out are deemed to have achieved a suitable content, as indicated by observation of the visualization agent disposed in the materials that flow out. For example, two fluent precursors associated with a blue dye are introduced into a uterus and pumped therein until the color of materials exiting the uterus indicates that unwanted fluids have been washed out of the uterus and the uterus is substantially full of the precursors.

An embodiment is a method of a user applying a hydrogel coating to a substrate and selecting a visually observable visualization agent to observe the hydrogel coating. The user may use visualization agents to see the hydrogel with the human eye or with the aid of an imaging device that detects visually observable visualization agents, e.g., a videocamera. A visually observable visualization agent is an agent that has a color detectable by a human eye. A characteristic of providing imaging to an X-ray or MRI machine is not a characteristic sufficient to establish function as a visually observable visualization agent.

A coating with a free surface may have surface that can be viewed for use with a visually observable visualization agent. In contrast, a hydrogel injected into a blood vessel, muscle, or other tissue has essentially no surface for viewing a visualization agent because its surface area is essentially engaged with tissues of the patient. Further, polymers injected into a tissue lack a surface that is disposed on the surface of a tissue and do not provide a means for a user to control the thickness of the coating on the surface of the tissue. Hydrogels that are merely injected into a patient's body would not be equivalent to embodiments of the present invention that involve a hydrogel coating on a substrate and are inoperative for embodiments of the invention that entail use of a visualization agent in a hydrogel coating.

An embodiment of the invention involves a mixture or a process of mixing hydrophilic reactive precursor species having nucleophilic functional groups with hydrophilic reactive precursor species having electrophilic functional groups such that they form a mixture that crosslinks quickly after contact with the tissue of a patient to form a biodegradable hydrogel that coats and adheres to a tissue. This may be achieved by making reactive precursor species that crosslink quickly after mixing. Hydrophilic reactive precursor species can be dissolved in buffered water such that they provide low viscosity solutions that readily mix and flow when contacting the tissue. As they flow across the tissue, they conform to the shape of the small features of the tissue such as bumps, crevices and any deviation from molecular smoothness. If the reactive precursor species are too slow to crosslink, they will flow off the tissue and away into other portions of the body with the result that the user will be unable to localize the hydrogel on the desired tissue. Without limiting the invention to a particular theory of operation, it is believed that reactive precursor species s that crosslink appropriately quickly after contacting a tissue surface will form a three dimensional structure that is mechanically interlocked with the tissue it is in contact with. This interlocking contributes to adherence, intimate contact, and essentially continuous coverage of the coated region of the tissue.

Suitable crosslinking times vary for different applications. In most applications, the crosslinking reaction leading to gelation occurs within about 10 minutes, more preferably within about 2 minutes, even more preferably within 10 seconds. In the case of most surgical adhesion prevention applications, it is preferable to use a hydrogel that crosslinks in less than about 10 seconds and more preferably in about 2-4 seconds in order to allow a user to make multiple passes with a hydrogel applicator tool such as a catheter. In the case of tissues that can be accessed only indirectly, longer times are most preferable to allow the gel a longer time to flow into the inaccessible space.

Functional Groups

A precursor may be multifunctional, meaning that it comprises two or more electrophilic or nucleophilic functional groups, such that a nucleophilic functional group on one precursor may react with an electrophilic functional group on another precursor to form a covalent bond. At least one of the precursors may comprise more than two functional groups, so that, as a result of electrophilic-nucleophilic reactions, the precursors combine to form crosslinked polymeric products. Such reactions are referred to as “crosslinking reactions”.

Preferably, each precursor comprises only nucleophilic or only electrophilic functional groups, so long as both nucleophilic and electrophilic precursors are used in the crosslinking reaction. Thus, for example, if a crosslinker has nucleophilic functional groups such as amines, the functional polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly(allyl amine), or amine-terminated di-or multifunctional poly(ethylene glycol) (“PEG”) can be used.

Water Soluble Cores

The precursors may have biologically inert and water soluble cores. When the core is a polymeric region that is water soluble, preferred polymers that may be used include: polyether, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly (vinyl pyrrolidinone) (“PVP”); poly (amino acids); dextran and proteins such as albumin. The polyethers and more particularly poly(oxyalkylenes) or poly(ethylene glycol) or polyethylene glycol are especially preferred. When the core is small molecular in nature, any of a variety of hydrophilic functionalities can be used to make the precursor water soluble. For example, functional groups like hydroxyl, amine, sulfonate and carboxylate, which are water soluble, maybe used to make the precursor water soluble. In addition, N-hydroxysuccinimide (“NHS”) ester of subaric acid is insoluble in water, but by adding a sulfonate group to the succinimide ring, the NHS ester of subaric acid may be made water soluble, without affecting its reactivity towards amine groups.

Biodegradable Linkages

If it is desired that the biocompatible crosslinked polymer be biodegradable or absorbable, one or more precursors having biodegradable linkages present in between the functional groups may be used. The biodegradable linkage optionally also may serve as the water soluble core of one or more of the precursors. In the alternative, or in addition, the functional groups of the precursors may be chosen such that the product of the reaction between them results in a biodegradable linkage. For each approach, biodegradable linkages may be chosen such that the resulting biodegradable biocompatible crosslinked polymer will degrade or be absorbed in a desired period of time. Preferably, biodegradable linkages are selected that degrade under physiological conditions into non-toxic products.

The biodegradable linkage may be chemically or enzymatically hydrolyzable or absorbable. Illustrative chemically hydrolyzable biodegradable linkages include polymers, copolymers and oligomers of glycolide, dl-lactide, l-lactide, caprolactone, dioxanone, and trimethylene carbonate. Illustrative enzymatically 5 hydrolyzable biodegradable linkages include peptidic linkages cleavable by metalloproteinases and collagenases. Additional illustrative biodegradable linkages include polymers and copolymers of poly(hydroxy acid)s, poly(orthocarbonate)s, poly(anhydride)s, poly(lactone)s, poly(aminoacid)s, poly(carbonate)s, and poly(phosphonate)s.

Visualization Agents

Where convenient, the biocompatible crosslinked polymer or precursor solutions (or both) may contain visualization agents to improve their visibility during surgical procedures. Visualization agents are especially useful when used in MIS procedures, due among other reasons to their improved visibility on a color monitor.

Visualization agents may be selected from among any of the various non-toxic colored substances suitable for use in medical implantable medical devices, such as FD&C BLUE dyes 3 and 6, eosin, methylene blue, indocyanine green, or colored dyes normally found in synthetic surgical sutures. The preferred color is green or blue because it has better visibility in presence of blood or on a pink or white tissue background. Red is the least preferred color, when used on a highly vascularized tissue that is red in color. However, red may be suitable when the underlying tissue is white, for example the cornea.

The visualization agent may be present with either reactive precursor species, e.g., a crosslinker or functional polymer solution. The preferred colored substance may or may not become chemically bound to the hydrogel. The visualization agent may be used in small quantities, preferably less than 1% weight/volume, more preferably less that 0.01% weight/volume and most preferably less than 0.001% weight/volume concentration. Additional visualization or imaging agents may be used, such as fluorescent (e.g., green or yellow fluorescent under visible light) compounds (e.g., fluorescein or eosin), x-ray contrast imaging agents (e.g., iodinated compounds) for visibility under x-ray imaging equipment, ultrasonic imaging contrast agents, or MRI imaging contrast agents (e.g., Gadolinium containing compounds).

Visually observable visualization agents are preferred for some embodiments. Wavelengths of light from about 400 to 750 nm are observable to the human as colors (R. K. Hobbie, Intermediate Physics for Medicine and Biology, 2^{nd} Ed., pages 371-373). Blue color is perceived when the eye receives light that is predominantly from about 450 to 500 nm in wavelength and green is perceived at about 500 to 570 nm (Id.). The color of an object is therefore determined by the predominant wavelength of light that it reflects or emits. Further, since the eye detects red or green or blue, a combination of these colors may be used to simulate any other color merely by causing the eye to receive the proportion of red, green, and blue that is perceived as the desired color by the human eye. Blue and green visualization agents are preferred since they are most readily visible when observing in situ crosslinking due to the approximately red color of the background color of tissue and blood. The color blue, as used herein, means the color that is perceived by a normal human eye stimulated by a wavelength of about 450 to 500 nm and the color green, as used herein, means the color that is perceived by a normal human eye stimulated by a wavelength of about 500 to 570 nm.

Crosslinking Reactions

The crosslinking reactions preferably occur in aqueous solution under physiological conditions. More preferably the crosslinking reactions occur “in situ”, meaning they occur at local sites such as on organs or tissues in a living animal or human body. More preferably the crosslinking reactions do not release heat of polymerization. Preferably the crosslinking reaction leading to gelation occurs within about 10 minutes, more preferably within about 2 minutes, more preferably within about one minute, and most preferably within about 30 seconds.

Certain functional groups, such as alcohols or carboxylic acids, do not normally react with other functional groups, such as amines, under physiological pH (e.g., pH 7.2-11.0, 37° C.). However, such functional groups can be made more reactive by using an activating group such as N-hydroxysuccinimide. Several methods for activating such functional groups are known in the art. Preferred activating groups include carbonyldiimidazole, sulfonyl chloride, aryl halides, sulfosuccinimidyl esters, N-hydroxysuccinimidyl ester, succinimidyl ester, epoxide, aldehyde, maleimides, imidoesters and the like. The N-hydroxysuccinimide esters or N-hydroxysulfosuccinimide groups are the most preferred groups for crosslinking of proteins or amine functionalized polymers such as amino terminated polyethylene glycol (“APEG”).

FIGS. 5 to 9 illustrate various embodiments of precursors, small molecule crosslinkers, and functional polymers. The term precursor encompasses small molecule crosslinkers and functional polymers. FIG. 1 illustrates possible configurations of degradable electrophilic crosslinkers or functional polymers. The biodegradable regions are represented by ( [symbol] ) the functional groups are represented by ( [symbol] ) and the inert water soluble cores are represented by ( [symbol] ). For crosslinkers, the central core is a water soluble small molecule and for functional polymers the central core is a water soluble polymer of natural or synthetic origin.

Structure A in FIG. 5 may be a linear water soluble and biodegradable functional polymer, end-capped with two functional groups (e.g., N-hydroxysuccinimide ester or NHS, epoxide or similar reactive groups). The water soluble core may be a polyalkylene oxide, preferably polyethylene glycol block copolymer, and it is extended with at least one biodegradable linkage between it and each terminal functional group. The biodegradable linkage may be a single linkage or copolymers or homopolymers of absorbable polymers such as polyhydroxy acids or polylactones.

Structure B in FIG. 5 may be a functional polymer that is a branched or star shaped biodegradable functional polymer which has an inert polymer at the center. Its inert and water soluble core may be terminated with oligomeric biodegradable extensions, which in turn may be terminated with reactive functional groups.

Structures C and D in FIG. 5 may multifunctional biodegradable polymers. This polymer may have a water-soluble soluble core at the center, which is a 4 arm, tetrafunctional polyethylene glycol (Structure C) or block copolymer of PEO-PPO-PEO such as TETRONIC 908 (Structure D) which may be extended with by small oligomeric extensions of biodegradable polymer to maintain water solubility and terminated with reactive functional end-groups such as CDI or NHS.

Structure E in FIG. 5 may be a multifunctional star or graft type biodegradable polymer. This polymer may be a water-soluble polymer like polyethylene oxide, polyvinyl alcohol or poly(vinyl pyrrolidinone) at the core which is completely or partially extended with biodegradable polymer. The biodegradable polymer may be terminated with reactive end groups.

Structures A-E in FIG. 5 need not have polymeric cores and may be small molecule crosslinkers. In that case, the core may comprise a small molecule like ethoxylated glycerol, inositol, trimethylolpropane etc. to form the resultant crosslinker. In addition, Structures A-E in FIG. 5 need not have polymeric biodegradable extensions, and the biodegradable extensions may consist of small molecules like succinate or glutarate or combinations of 2 or more esters, such as glycolate/2-hydroxybutyrate or glycolate/4-hydroxyproline, etc. A dimer or trimer of 4-hydroxyproline may be used not only to add degradability, but also to add nucleophilic functional group reactive sites via the pendant primary amines which are part of the hydroxyproline moiety.

Other variations of the core, the biodegradable linkage, and the terminal electrophilic group in Structures A-E in FIG. 5 may be constructed, so long as the resulting functional polymer has the properties of low tissue toxicity, water solubility, and reactivity with nucleophilic functional groups.

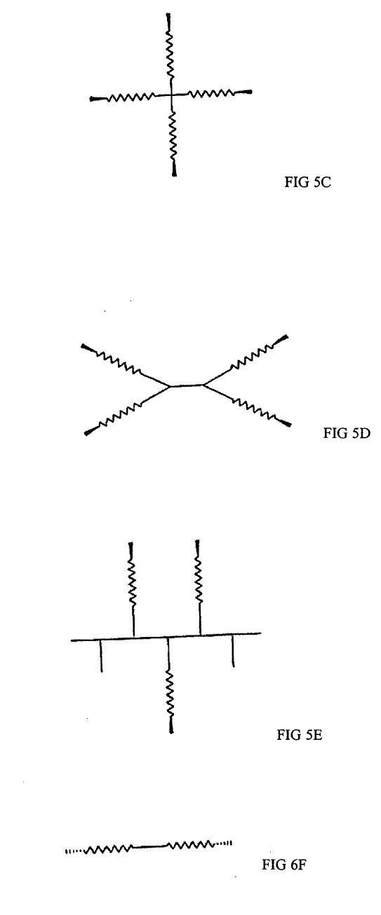

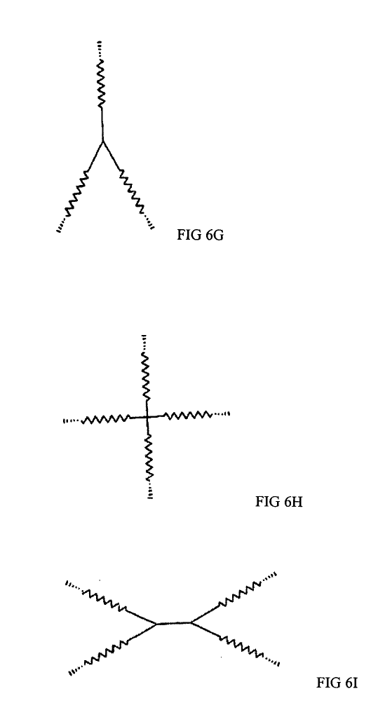

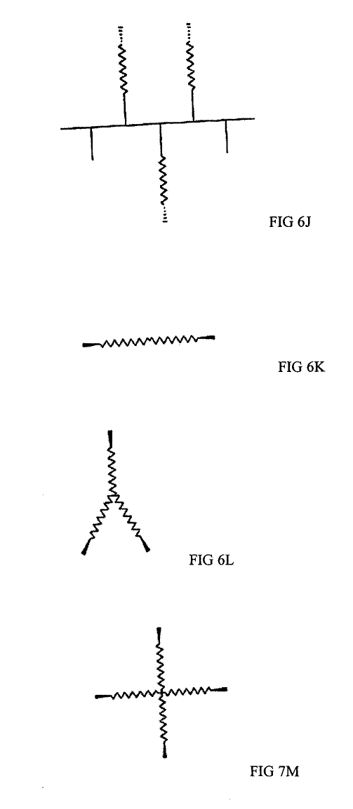

FIG. 6 illustrates various embodiments of nucleophilic biodegradable water soluble crosslinkers and functional polymers suitable for use with electrophilic functional polymers and crosslinkers described herein.

The biodegradable regions are represented by 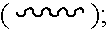
the functional groups are represented by 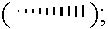
and the inert water soluble cores are represented by ( [symbol] ). For crosslinkers, the central core is a water soluble small molecule and for functional polymers the central core is a water soluble polymer of natural or synthetic origin.

Structure F in FIG. 6 may be a linear water soluble biodegradable polymer terminated with reactive functional groups like primary amine. The linear water-soluble core may be a polyalkylene oxide, preferably polyethylene glycol block copolymer, which may be extended with the biodegradable region which may be a copolymer or homopolymer of polyhydroxy acids or polylactones. This biodegradable polymer may be terminated with primary amines.

Structure G in FIG. 6 may be a branched or star shaped biodegradable polymer which has an inert polymer at the center. The inert polymer may be extended with single or oligomeric biodegradable extensions which may be terminated with reactive functional groups.

Structures H and I in FIG. 6 may be multifunctional 4 arm biodegradable polymers. These polymers again may have water-soluble cores at their center which are either a 4 arm, tetrafunctional polyethylene glycol (Structure H) or a block copolymer of PEO-PPO-PEO such as TETRONIC 908 (Structure I), extended with small oligomeric extensions of biodegradable polymers to maintain water solubility, and terminated with functional groups such as amines and thiols.

Structure J in FIG. 6 may be a multifunctional star or graft type biodegradable polymer. This polymer has a water soluble polymer like polyethylene oxide, polyvinyl alcohol or poly(vinyl pyrrolidinone) at the core which is completely or partially extended with biodegradable polymer. The biodegradable polymer may be terminated with reactive end groups.

Structures F-J in FIG. 6 need not have polymeric cores and may be small molecule crosslinkers. In that case, the core may comprise a small molecule like ethoxylated glycerol, inositol, trimethylolpropane etc. to form the resultant crosslinker.

Other variations of the core, the biodegradable linkage, and the terminal nucleophilic functional group in Structures F-J in FIG. 6 may be constructed with the resulting functional polymer has the properties of low tissue toxicity, water solubility, and reactivity with electrophilic functional groups.

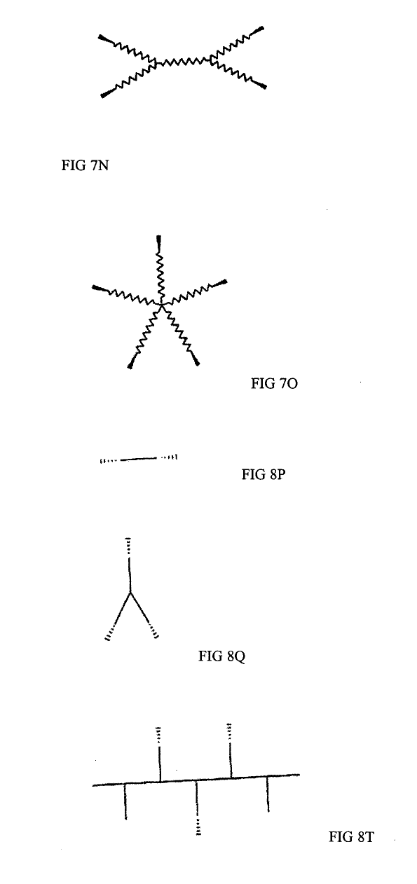

FIG. 7 illustrates configurations of water-soluble electrophilic crosslinkers or functional polymers where the core is biodegradable. The biodegradable regions are represented by 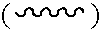
and the functional groups are represented by 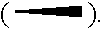
The biodegradable core is terminated with a reactive functional group that is also water solubilizing, such a N-hydroxysulfosuccinimide ester (“SNHS”) or N-hydroxyethoxylated succinimide ester (“ENHS”).

Structure K in FIG. 7 depicts a difunctional biodegradable polymer or oligomer terminated with SNHS or ENHS. The oligomers and polymers may be made of a poly(hydroxy acid) such as poly(lactic acid), which is insoluble in water. However, the terminal carboxylic acid group of these oligomers or polymers can be activated with N-hydroxysulfosuccinimide ester (“SNHS”) or N-hydroxyethoxylated succinimide ester (“ENHS”) groups. An ionic group, like a metal salt (preferably sodium salt) of sulfonic acid, or a nonionic group, like a polyethylene oxide on the succinimide ring, provides water-solubility while the NHS ester provides chemical reactivity towards amines. The sulfonate groups (sodium salts) or ethoxylated groups on the succinimide ring solubilize the oligomer or polymer without appreciably inhibiting reactivity towards amine groups.

Structures L-O in FIG. 7 represent multi-branched or graft type structures with terminal SNHS or ENHS group. The cores may comprise various non-toxic polyhydroxy compounds like sugars (xylitol, erythritol), glycerol, trimethylolpropane, which have been reacted with anhydrides such as succinic or glutaric anhydrides. The resultant acid groups were then activated with SNHS or ENHS groups to form water soluble crosslinkers or functional polymers.

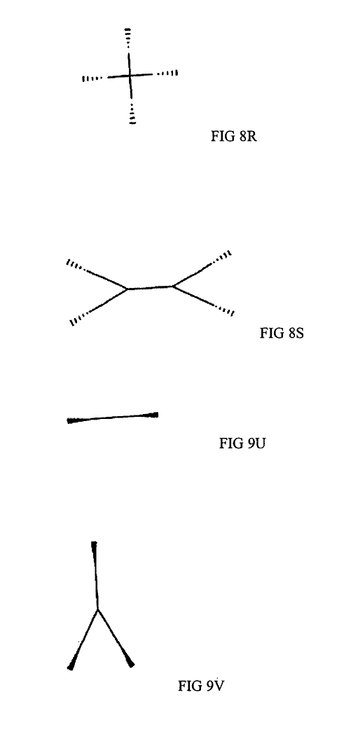

FIG. 8 illustrates various nucleophilic functional polymers or crosslinkers that are not biodegradable. The nucleophilic functional groups are represented by ( [symbol] ) and the inert water-soluble cores are represented by ( [symbol] ). For crosslinkers, the central core is a water-soluble small molecule and for functional polymers the central core is a water soluble polymer of natural or synthetic origin.

When Structure P in FIG. 8 is a functional polymer it may be a water-soluble linear polymer such as polyethylene glycol terminated with reactive end group such as primary amines and thiols. Such polymers are commercially available from Sigma (Milwaukee, Wis.) and Shearwater Polymers (Huntsville, Ala.). Some other preferred difunctional polymers are PPO-PEO-PPO block copolymers such as PLURONIC F68 terminated with amine groups. Pluronic or TETRONIC polymers are normally available with terminal hydroxyl groups. The hydroxyl groups are converted into amine groups by methods known in the art.

Structures Q-T in FIG. 8 may be functional polymers they may be multifunctional graft or branch type water soluble copolymers with terminal amine groups. Structures P-T in FIG. 8 need not have polymeric cores and may be small molecule crosslinkers. In that case, the core may comprise a small molecule like ethoxylated glycerol, inositol, trimethylolpropane, dilysine etc. to form the resultant crosslinker.

Other variations of the core and the terminal nucleophilic functional group in Structure P-T in FIG. 8 may be employed with the properties of low tissue toxicity, water solubility, and reactivity with electrophilic functional groups maintained.

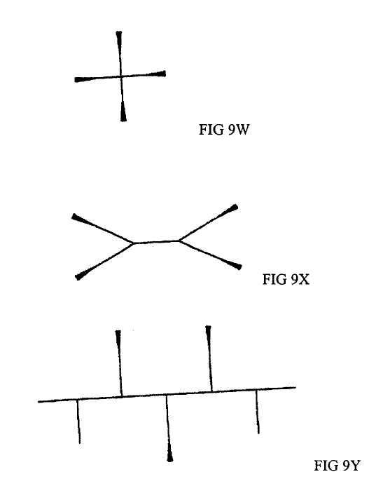

FIG. 9 illustrates various electrophilic functional polymers or crosslinkers that are not biodegradable. The electrophilic functional groups are represented by 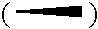
and the inert water soluble cores are represented by ( [symbol] ). For crosslinkers, the central core is a water soluble small molecule and for functional polymers the central core is a water soluble polymer of natural or synthetic origin.

When Structure U is a functional polymer, it may be a water-soluble polymer such as polyethylene glycol terminated reactive end group such as NHS or epoxide. Such polymers are commercially available from Sigma and Shearwater polymers. Some other preferred polymers are PPO-PEO-PPO block copolymers such as PLURONIC F68 terminated with NHS or SNHS group. PLURONIC or TETRONIC polymers are normally available with terminal hydroxyl groups. The hydroxyl groups are converted into acid group by reacting with succinic anhydride. The terminated acid groups are reacted with N-hydroxysuccinimide in presence of DCC to generate NHS activated PLURONIC polymer. When Structures V-Y are functional polymers they may be multifunctional graft or branch type PEO or PEO block copolymers (TETRONICS) activated with terminal reactive groups such as NHS.

Structures U-Y in FIG. 9 need not have polymeric cores and may be small molecule crosslinkers. In that case, the core may comprise a small molecule like ethoxylated glycerol, tetraglycerol, hexaglycerol, inositol, trimethylolpropane, dilysine etc. to form the resultant crosslinker. Other variations of the core and the terminal nucleophilic functional group in Structures U-Y in FIG. 5 may be employed, so long as the properties of low tissue toxicity, water solubility, and reactivity with electrophilic functional groups are maintained.

B. Preparation of Precursors

The precursors may be prepared using variety of synthetic methods. Certain compositions are described in Table 1.

TABLE 1
Select Precursors

| Structure | Brief Description | Typical Example |
|---|---|---|
| A | Water soluble, linear | Polyethylene glycol or ethoxylated |
| | difunctional crosslinker or | propylene glycol chain extended with |
| | functional polymer with water | oligolactate and terminated with N- |
| | soluble core, extended with | hydroxysuccinimide esters |
| | biodegradable regions such as | |
| | oligomers of hydroxyacids or | |
| | peptide sequences which are | |
| | cleavable by enzymes and | |
| | terminated with protein reactive | |
| | functional groups | |
| B | Water soluble, trifuncational | Ethoxylated glycerol chain extended |
| | crosslinker or functional | with oligolactate and terminated with |
| | polymer with water soluble core, | N-hydroxysuccinimide esters |
| | extended with biodegradable | |
| | regions such as oligomers of | |
| | hydroxyacids or peptide | |
| | sequences and terminated with | |
| | protein reactive functional | |
| | groups | |
| C | Water soluble, tetrafunctional | 4 arm polyethylene glycol, erythritol |
| | crosslinker or functional | or pentaerythritol or pentaerythritol |
| | polymer with water soluble core, | chain extended with oligolactate and |
| | extended with biodegradable | terminated with N- |
| | regions such as oligomers of | hydroxysuccinimide esters |
| | hydroxyacids or peptide | |
| | sequences and terminated with | |
| | protein reactive functional | |
| | groups | |
| D | Water soluble, tetrafunctional | Ethoxylated ethylene diamine or |
| | crosslinker or functional | polyethylene oxide-polypropylene |
| | polymer with water soluble core, | oxide-polyethylene oxide block |
| | extended with biodegradable | copolymer like TETRONIC 908 |
| | regions such as oligomers of | chain extended with |
| | hydroxyacids or peptide | oligotrimethylene carbonate and |
| | sequences and terminated with | terminated with N- |
| | protein reactive functional | hydroxysuccinimide ester |
| | groups | |
| E | Water soluble, branched | Low molecular weight polyvinyl |
| | crosslinker or functional | alcohol with 1% to 20% hydroxyl |
| | polymer with water soluble core, | groups extended with oligolactate and |
| | extended with biodegradable | terminated with N- |
| | regions such as oligomers of | hydroxysuccinimide ester |
| | hydroxyacids or peptide | |
| | sequences and terminated with | |
| | protein reactive functional | |
| | groups | |
| F | Water soluble, liner difunctional | Polyethylene oxide-polypropylene |
| | crosslinker or functional | oxide-polyethylene oxide block |
| | polymer with water soluble core, | copolymer surfactant like |
| | extended with biodegradable | PLURONIC F68 chain extended with |
| | regions such as oligomers of | oligolactate and terminated with |
| | hydroxyacids or peptide | amino acids such as lysine or peptide |
| | sequences and terminated with | sequences that may contain two |
| | amines, carboxylic acid or thiols | amine groups |
| G | Water soluble, trifunctional | Ethoxylated glycerol chain extended |
| | crosslinker or functional | with oligolactate and terminated with |
| | polymer with water soluble core, | aminoacid such as lysine |
| | extended with biodegradable | |
| | regions such as oligomers of | |
| | hydroxyacids or peptide | |
| | sequences and terminated with | |
| | amines, carboxylic acid or thiols | |
| H | Water soluble, tetrafuncational | 4 arm polyethylene glycol or tetraerythritol |
| | crosslinker or functional | chain extended with |
| | polymer with water soluble core, | oligolactate and terminated with |
| | extended with biodegradable | aminoacid such as lysine |
| | regions such as oligomers of | |
| | hydroxyacids or peptide | |
| | sequences and terminated with | |
| | amines, carboxylic acid or thiols | |
| I | Water soluble, tetrafunctional | Ethoxylated ethylene diamine or |
| | crosslinker or functional | polyethylene oxide-polypropylene |
| | polymer with water soluble core, | oxide-polyethylene oxide block |
| | extended with biodegradable | copolymer like TETRONIC 908 |
| | regions such as oligomers of | chain extended with |
| | hydroxyacids or peptide | oligotrimethylene carbonate and |
| | sequences and terminated with | terminated with aminoacid such as |
| | amines, carboxylic acid or thiols | lysine |
| J | Water soluble, multifunctional or | Low molecular weight polyvinyl |
| | graft type crosslinker or | alcohol with 1-20% hydroxyl groups |
| | functional polymer with water | extended with oligolactate and |
| | soluble core, extended with | terminated with aminoacid such as |
| | biodegradable regions such as | lysine |
| | oligomers of hydroxyacids or | |
| | peptide sequences and | |
| | terminated with amines, | |
| | carboxylic acid or thiols | |
| K | Water soluble, linear | Difunctional oligolactic acid with |
| | difunctional crosslinker or | terminal carboxyl groups which are |
| | functional polymer such as | activated with n- |
| | oligomers of hydroxyacids or | hydroxysulfosuccinimi de ester or |
| | peptide sequences which are | ethoxylated n-hydroxysuccinimide |
| | terminated with protein reactive | ester. |
| | functional groups | |
| L | Water soluble branched | Trifunctional oligocaprolactone with |
| | trifunctional crosslinker or | terminal carboxyl groups which are |
| | functional polymer such as | activated with n- |
| | oligomers of hydroxyacids or | hydroxysulfosuccinimi de ester or |
| | peptide sequences which are | ethoxylated n-hydroxysuccinimide |
| | terminated with protein reactive | ester. |
| | functional groups | |
| M | Water soluble, branched | Tetrafunctional oligocaprolactone |
| | tetrafunctional crosslinker or | with terminal carboxyl groups which |
| | functional polymer such as | are activated with n- |
| | oligomers of hydroxyacids or | hydroxysulfosuccinimi de ester or |
| | peptide sequences which are | ethoxylated n-hydroxysuccinimide |
| | terminated with protein reactive | ester. |
| | functional groups | |
| N | Water soluble, branched | Tetrafunctional oligocaprolatone with |
| | tetrafunctional crosslinker or | terminal carboxyl groups which are |
| | functional polymer such as | activated with n- |
| | oligomers of hydroxyacids or | hydroxysulfosuccinimi de ester or |
| | peptide sequences which are | ethoxylated n-hydroxysuccinimide |
| | terminated with protein reactive | ester. |
| | functional groups | |
| O | Water soluble, branched | Multifunctional oligolactic acid with |
| | multifunctional crosslinker or | terminal carboxyl groups which are |
| | functional polymer such as | activated with n- |
| | oligomers f hydroxyacids or | hydroxysulfosuccinimi de ester or |
| | peptide sequences which are | ethoxylated n-hydroxysuccinimide |
| | terminated with protein reactive | ester. |
| | functional groups | |
| P | Water soluble, linear | Polyethylene glycol with terminal |
| | difunctional crosslinker or | amines groups |
| | functional polymer terminated | |
| | with amines, carboxylic acid or | |
| | thiols functional groups | |
| Q | Water soluble, branched | Ethoxylated glycerol with terminal |
| | trifunctional crosslinker or | amines groups |
| | functional polymer terminated | |
| | with amines, carboxylic acid or | |
| | thiols as functional group | |
| R | Water soluble, branched | 4 arm polyethylene glycol modified |
| | tetrafunctional crosslinker of | to produce terminal amine groups |
| | functional polymer terminated | |
| | with amines, carboxylic acid or | |
| | thiols functional groups | |
| S | Water soluble, branched | Ethoxylated ethylene diamine or |
| | tetrafunctional crosslinker or | polyethylene oxide-polyprophylene |
| | functional polymer terminated | oxide-polyethylene oxide block |
| | with amines, carboxylic acid or | copolymer like TETRONIC 908 |
| | thiols functional groups | modified to generate terminal amine |
| | | groups |
| T | Water soluble, branched or graft | Polylysine, albumin, polyallyl amine |
| | crosslinker or functional | |
| | polymer with terminal amines, | |
| | carboxylic acid or thiols | |
| | functional groups | |
| U | Water soluble, linear | Polylysine, albumin, polyallyl amine |
| | difunctional crosslinker or | |
| | functional polymer terminated | |
| | with protein reactive functional | |
| | groups | |
| V | Water soluble branched | Ethoxylated glycerol terminated with |
| | trifunctional crosslinker or | n-hydroxysuccinimide |
| | functional polymer terminated | |
| | with protein reactive functional | |
| | groups | |
| W | Water soluble branched | 4 arm polyethylene glycol terminated |
| | tetrafunctional crosslinker or | with n-hydroxysuccinimide esters |
| | functional polymer terminated | |
| | with protein reactive functional | |
| | groups | |
| X | Water soluble branched | Ethoxylated ethylene diamine or |
| | tetrafunctional crosslinker or | polyethylene oxide-polypropylene |
| | functional polymer terminated | oxide-polyethylene oxide block |
| | with protein reactive functional | copolymer like TETRONIC 908 with |
| | groups | n-hydroxysuccinimide ester as end |
| | | group |
| Y | Water soluble, branched or graft | Poly (vinyl pyrrolidinone)-co-poly |
| | polymer crosslinker or | (n-hydroxysuccinimide acrylate) |
| | functional polymer with protein | copolymer (9:1), molecular weight <40000 Da |
| | reactive functional groups | |

The biodegradable links of precursor Structures A-J in FIGS. 5 and 6 may be composed of specific di or multifunctional synthetic amino acid sequences which are recognized and cleaved by enzymes such as collagenase, and may be synthesized using methods known to those skilled in the peptide synthesis art. For example, Structures A-E in FIG. 5 may be obtained by first using carboxyl, amine or hydroxy terminated polyethylene glycol as a starting material for building a suitable peptide sequence. The terminal end of the peptide sequence is converted into a carboxylic acid by reacting succinic anhydride with an appropriate amino acid. The acid group generated is converted to an NHS ester by reaction with N-hydroxysuccinimide.

The functional polymers described in FIG. 6 may be prepared using a variety of synthetic methods. In a preferred embodiment, the polymer shown as Structure F may be obtained by ring opening polymerization of cyclic lactones or carbonates initiated by a dihydroxy compound such as PLURONIC F 68 in the presence of a suitable catalyst such as stannous 2-ethylhexanoate. The molar equivalent ratio of caprolactone to PLURONIC is kept below 10 to obtain a low molecular weight chain extension product so as to maintain water solubility. The terminal hydroxyl groups of the resultant copolymer are converted into amine or thiol by methods known in the art.

In a preferred method, the hydroxyl groups of a PLURONIC-caprolactone copolymer are activated using tresyl chloride. The activated groups are then reacted with lysine to produce lysine terminated PLURONIC-caprolactone copolymer. Alternatively, an amine-blocked lysine derivative is reacted with the hydroxyl groups of a PLURONIC-caprolactone copolymer and then the amine groups are regenerated using a suitable deblocking reaction.

Structures G, H, I and J in FIG. 6 may represent multifunctional branched or graft type copolymers having water soluble core extended with oligohydroxy acid polymer and terminated with amine or thiol groups.

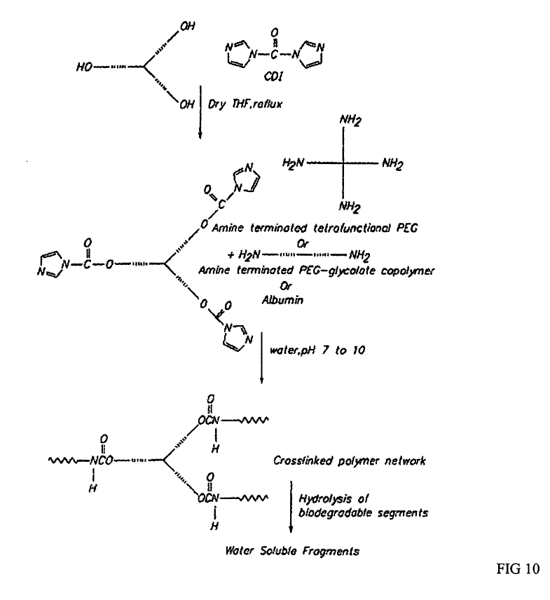

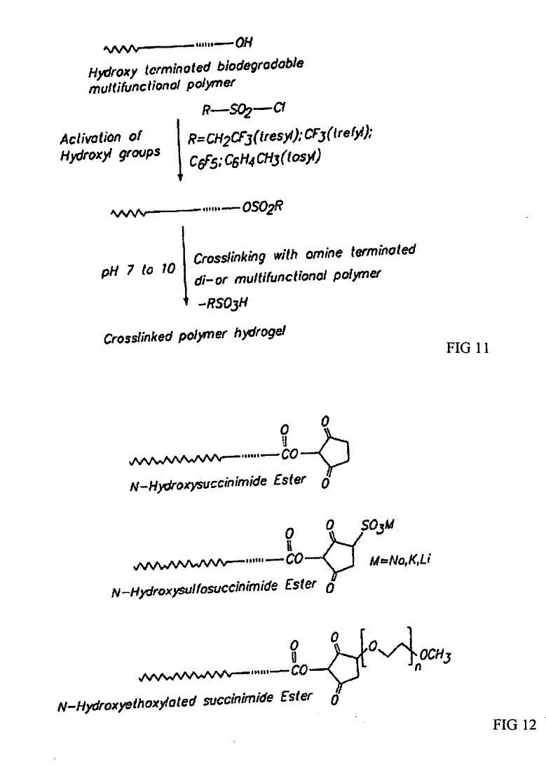

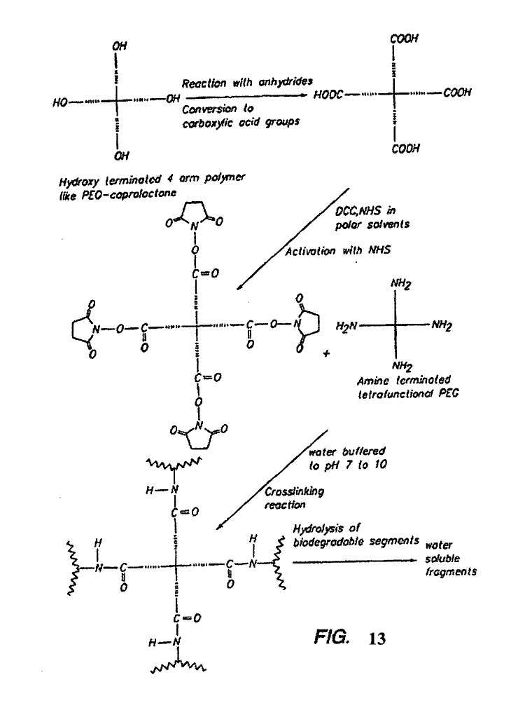

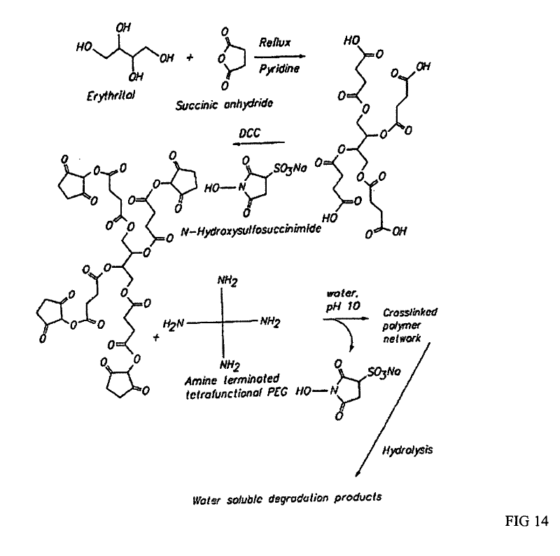

For example, in a preferred embodiment, the functional polymer illustrated as Structure G in FIG. 6 is obtained by ring opening polymerization of cyclic lactones or carbonates initiated by a tetrahydroxy compound such as 4 arm, tetrahydroxy polyethylene glycol (molecular weight 10,000 Da), in the presence of a suitable catalyst such as stannous octoate. The molar equivalent ratio of cyclic lactone or carbonate to PEG is kept below 10 to obtain a low molecular weight extension, and to maintain water solubility (polymers of cyclic lactones generally are not as water soluble as PEG). Alternatively, hydroxyacid as a biodegradable link may be attached to the PEG chain using blocking/deblocking chemistry known in the peptide synthesis art. The terminal hydroxy groups of the resultant copolymer are activated using a variety of reactive groups known in the art. The CDI activation chemistry and sulfonyl chloride activation chemistry is shown in FIGS. 10 and 11, respectively.

Some preferred reactive groups are N-hydroxysuccinimide esters, synthesized by any of several methods. In a preferred method, hydroxyl groups are converted to carboxylic groups by reacting them with anhydrides such as succinic anhydride in the presence of tertiary amines such as pyridine or triethylamine or dimethylaminopyridine (“DMAP”). Other anhydrides such as glutaric anhydride, phthalic anhydride, maleic anhydride and the like may also be used. The resultant terminal carboxyl groups are reacted with N-hydroxysuccinimide in the presence of dicyclohexylcarbodiimide (“DCC”) to produce N-hydroxysuccinimide ester (referred as NHS activation). The NHS activation and crosslinking reaction scheme is shown in FIG. 8. Some preferred N-hydroxysuccinimide esters are shown in FIG. 13.

In an embodiment, the polymer shown as structure H is obtained by ring opening polymerization of glycolide or trimethylene carbonate initiated by a tetrahydroxy compound such as tetrafunctional polyethylene glycol (molecular weight 2000 Da) in the presence of a catalyst such as stannous 2-ethylhexoate. The molar equivalent ratio of glycolide to PEG is kept from 2 to 10 to obtain a low molecular weight extension. The terminal hydroxy groups of the resultant copolymer are converted into amine groups by reaction with lysine as mentioned previously. Similar embodiments can be obtained using analogous chain extension synthetic strategies to obtain structures F, G, I and J by starting with the appropriate corresponding polyol.

Structures K, L, M, N and O in FIG. 7 are made using a variety of synthetic methods. In a preferred embodiment, the polymer shown as Structure L in FIG. 7 is obtained by ring opening polymerization of cyclic lactones by a trihydroxy compound such as glycerol in the presence of a catalyst such as stannous 2-ethylhexanoate. The molar equivalent ratio of cyclic lactone to glycerol is kept below 2, so that only low molecular weight oligomers are obtained. The low molecular weight oligomer ester is insoluble in water. The terminal hydroxy groups of the resultant copolymer are activated using N-hydroxysulfosuccinimide groups. This is achieved by converting hydroxy groups to carboxylic groups by reacting with anhydrides such as succinic anhydride in presence of tertiary amines. The resultant terminal carboxyl groups are reacted with N-hydroxysulfosuccinimide or N-hydroxyethoxylated succinimide in the presence of dicyclohexylcarbodiimide (“DCC”) to produce a sulfonated or ethoxylated NHS ester. The sulfonate or PEO chain on the succinimide ring gives water solubility to the oligoester.

The foregoing method generally is applied to solubilize only low molecular weight multi-branched oligoesters, with molecular weights below 1000. In another variation of this method, various non-toxic polyhydroxy compounds, preferably sugars, such as erythritol, xylitol are reacted with succinic anhydride in the presence of a tertiary amine. The terminal carboxyl group of succinated erythritol is esterified with N-hydroxysulfosuccinimide (FIG. 13). Similar embodiments may be obtained using analogous synthetic strategies to obtain structures K, and M-O by starting with the appropriate starting materials.

Structures P-R may be synthesized by reacting the appropriate starting material, such as a linear (P) or 2- or 3-arm branched PEG (Q, R) with hydroxy end groups, with lysine as mentioned previously, such that the arms of the PEG oligomers are capped with amine end groups. Structure S may be synthesized, using a multistep reaction, from PEG, glycerol and a diisocyanate. In the first step a PEG diol is reacted with excess diisocyanate, such as 4,4′diphenyl methane diisocyanate (“MDI”), methylene-bis (4-cyclohexylisocyanate) (“HMDI”) or hexamethylenediisocyanate (“HDI”). After purification the resultant PEG diisocyanate is added dropwise to excess glycerol or trimethylol propane or other triol and reacted to completion. The purified product, now having diol end groups, is again reacted with excess diisocyanate and purified, yielding a PEG-tetra-isocyanate. This tetrafunctional PEG subsequently may be reacted with excess PEG diols, yielding a 4 arm PEG synthesized from a PEG diol oligomer. In the final step lysine end groups are incorporated, as discussed previously.

Structure T may be synthesized as follows: First synthesize a random copolymer of PEG-monoacrylate and some other acrylate or combination of acrylates, such that the final polyacrylate is water soluble. Other acrylates include, but are not limited to, 2-hydroxyethylacrylate, acrylic acid, and acrylamide. Conditions may be varied to control the molecular weight as desired. In the final step, the acrylate is reacted with lysine as discussed previously, using an appropriate quantity to achieve the desired degree of amination.

One method of synthesizing Structures U-Y is to use dicyclohexylcarbodiimide coupling to a carboxylate end group. For Structures U-W, one can react the appropriate PEG-diol, -triol or -tetra-hydroxy starting material with excess succinic anhydride or glutaric anhydride such that all end groups are effectively carboxylated. Structures X and Y may be made in a manner similar to that used for Structures S and T, except that in the last step instead of end capping with lysine, end capping with succinic anhydride or glutaric anhydride is performed.

Preparation of Biocompatible Polymers

Several biocompatible crosslinked hydrogels may be produced using the precursors described in FIGS. 5 to 9. Certain preferred combinations of polymers suitable for producing such biocompatible crosslinked polymers are described in Table 2. In Table 2, the crosslinker functional groups are N-hydroxy succinimide esters and the functional polymer functional groups are primary amines.

TABLE 2
Biocompatible Polymers Synthesized from components of Table 1

| | Functional | | |
|---|---|---|---|
| Crosslinker | Polymer | | |
| Structure | Structure | Concentration | Medium |
| B or C | H and R | Molar Equivalent; >20% W/V | Borate or triethanol amine |
| | | | buffer, pH 7-10 |
| A, B or C | H, P, Q, R and S | Molar Equivalent; >20% W/V | Borate or triethanol amine |
| | | | buffer, pH 7-10 |
| Y | T, H, P and Q | Molar Equivalent; >10% W/V | Borate or triethanol amine |
| | | | buffer, pH 7-910 |
| W, V | H and J | Molar Equivalent; >20% W/V | Bicarbonate buffer, pH 7-10 |
| X | I, J and H | Molar Equivalent; >20% W/V | Borate or triethanol amine |
| | | | buffer, pH 7-10 |

The reaction conditions for crosslinking will depend on the nature of the functional groups. Preferred reactions are conducted in buffered aqueous solutions at pH 5 to 12. The preferred buffers are sodium borate buffer (pH 10) and triethanol amine buffer (pH 7). Elevated pH increases the speed of electrophilic-nucleophilic reactions. In some embodiments, organic solvents such as ethanol or isopropanol may be added to improve the reaction speed or to adjust the viscosity of a given formulation.

The synthetic crosslinked gels described above degrade due to hydrolysis of the biodegradable region. The degradation of gels containing synthetic peptide sequences will depend on the specific enzyme and its concentration. In some cases, a specific enzyme may be added during the crosslinking reaction to accelerate the degradation process.

When the crosslinker and functional polymers are synthetic (for example, when they are based on polyalkylene oxide), then it is desirable and in some cases essential to use molar equivalent quantities of the reactants. In some cases, molar excess crosslinker may be added to compensate for side reactions such as reactions due to hydrolysis of the functional group.

When choosing the crosslinker and crosslinkable polymer, at least one of polymers must have more than 2 functional groups per molecule and at least one degradable region, if it is desired that the resultant biocompatible crosslinked polymer be biodegradable. For example, the difunctional crosslinker shown as Structure A in FIG. 5 cannot form a crosslinked network with the difunctional polymers shown as Structure F in FIG. 6 or Structure P in FIG. 8. Generally, it is preferred that each biocompatible crosslinked polymer precursor have more than 2 and more preferably 4 or more functional groups.

Preferred electrophilic functional groups are NHS, SNHS and ENHS (FIG. 13). Preferred nucleophilic functional groups are primary amines. The advantage of the NHS-amine reaction is that the reaction kinetics lead to quick gelation usually within 10 about minutes, more usually within about 1 minute and most usually within about 10 seconds. This fast gelation is typically preferred for in situ reactions on live tissue.

The NHS-amine crosslinking reaction leads to formation of N-hydroxysuccinimide as a side product. The sulfonated or ethoxylated forms of N-hydroxysuccinimide are preferred due to their increased solubility in water and hence their rapid clearance from the body. The sulfonic acid salt on the succinimide ring does not alter the reactivity of NHS group with the primary amines.

The NHS-amine crosslinking reaction may be carried out in aqueous solutions and in the presence of buffers. The preferred buffers are phosphate buffer (pH 5.0-7.5), triethanolamine buffer (pH 7.5-9.0), borate buffer (pH 9.0-12), and sodium bicarbonate buffer (pH 9.0-10.0).

Aqueous solutions of NHS based crosslinkers and functional polymers preferably are made just before the crosslinking reaction due to reaction of NHS groups with water. Longer “pot life” may be obtained by keeping these solutions at lower pH (pH 4-5).

The crosslinking density of the resultant biocompatible crosslinked polymer is controlled by the overall molecular weight of the crosslinker and functional polymer and the number of functional groups available per molecule. A lower molecular weight between crosslinks such as 600 will give much higher crosslinking density as compared to a higher molecular weight such as 10,000. Higher molecular weight functional polymers are preferred, preferably more than 3000 so as to obtain elastic gels.

The crosslinking density also may be controlled by the overall percent solids of the crosslinker and functional polymer solutions. Increasing the percent solids increases the probability that an electrophilic functional group will combine with a nucleophilic functional group prior to inactivation by hydrolysis. Yet another method to control crosslink density is by adjusting the stoichiometry of nucleophilic functional groups to electrophilic functional groups. A one to one ratio leads to the highest crosslink density.

Preparation of Biodegradable Polymers

The biodegradable crosslinkers described in FIGS. 5 and 7 may be reacted with proteins, such as albumin, other serum proteins, or serum concentrates to generate crosslinked polymeric networks. Briefly, aqueous solutions of the crosslinkers described in FIG. 5 and FIG. 7 (at a concentration of 50 to 300 mg/ml) are mixed with concentrated solutions of albumin (600 mg/ml) to produce a crosslinked hydrogel. This reaction can be accelerated if a buffering agent, e.g., borate buffer or triethanol amine, is added during the crosslinking step.

The resultant crosslinked hydrogel is a semisynthetic hydrogel whose degradation depends on the degradable segment in the crosslinker as well as degradation of albumin by enzymes. In the absence of any degradable enzymes, the crosslinked polymer will degrade solely by the hydrolysis of the biodegradable segment. If polyglycolate is used as the biodegradable segment, the crosslinked polymer will degrade in 1-30 days depending on the crosslinking density of the network. Similarly, a polycaprolactone based crosslinked network will degrade in 1-8 months. The degradation time generally varies according to the type of degradable segment used, in the following order: polyglycolate<polylactate<polytrimethylene carbonate<polycaprolactone. Thus it is possible to construct a hydrogel with a desired degradation profile, from a few days to months, using a proper degradable segment.

The hydrophobicity generated by biodegradable blocks such as oligohydroxy acid blocks or the hydrophobicity of PPO blocks in PLURONIC or TETRONIC polymers are helpful in dissolving small organic drug molecules. Other properties which will be affected by incorporation of biodegradable or hydrophobic blocks are: water absorption, mechanical properties and thermosensitivity.

Methods of Using Biocompatible Polymers

The biocompatible crosslinked polymers and their precursors described above may be used in a variety of applications, such as components of tissue adhesives, tissue sealants, drug delivery vehicles, wound covering agents, barriers in preventing postoperative adhesions, and others. These and other suitable applications are reviewed in Schlag and Redl, “Fibrin Sealant” in Operative Surgery, volumes 1-7 (1986), which is incorporated herein by reference.

In Situ Formation

In many applications, the biocompatible crosslinked polymers of this invention typically will be formed “in situ” at a surgical site in the body. The various methodologies and devices for performing “in situ” gelation, developed for other adhesive or sealant systems such fibrin glue or sealant applications, may be used with the biocompatible crosslinked polymers of this invention. Thus, in one embodiment, an aqueous solution of a freshly prepared crosslinker (e.g., SNHS-terminated oligolactide synthesized from a glycerol core in phosphate buffered saline (“PBS”) at pH 5 to 7.2) and a functional polymer (e.g., albumin or amine terminated tetrafunctional polyethylene glycol at pH 10 in sodium borate) are applied and mixed on the tissue using a double barrel syringe (one syringe for each solution). The two solutions may be applied simultaneously or sequentially. In some embodiments, it is preferred to apply the precursor solutions sequentially so as to “prime” the tissue, resulting in improved adherence of the biocompatible crosslinked polymer to the tissue. Where the tissue is primed, the crosslinker precursor is preferably applied to the tissue first, followed by the functional polymer solution.

One may use specialized devices to apply the precursor solutions, such as those described in U.S. Pat. Nos. 4,874,368; 4,631,055; 4,735,616; 4,359,049; 4,978,336; 5,116,315; 4,902,281; 4,932,942; Published Patent Cooperation Treaty Patent Application No. WO 91/09641; and R. A. Tange, “Fibrin Sealant” in Operative Medicine: Otolaryngology, volume 1 (1986), the disclosures of which are herein incorporated by reference.

Drug Delivery

The subject crosslinkers, functional polymer and their reaction products, the crosslinked materials advantageously may be used for localized drug therapy. Biologically active agents or drug compounds that may be added and delivered from the crosslinked polymer or gel include: proteins, glycosaminoglycans, carbohydrates, nucleic acid, inorganic and organic biologically active compounds where specific biologically active agents include but are not limited to: enzymes, antibiotics, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides.

To prepare such crosslinked composition, the bioactive compounds described above are mixed with the crosslinkable polymer prior to making the aqueous solution or during the aseptic manufacturing of the functional polymer. This mixture then is mixed with the crosslinker to produce a crosslinked material in which the biologically active substance is entrapped. Functional polymers made from inert polymers like PLURONIC, TETRONICS or Tween surfactants are preferred in releasing small molecule hydrophobic drugs.

In a preferred embodiment, the active agent or agents are present in a separate phase when crosslinker and crosslinkable polymers are reacted to produce a crosslinked polymer network or gel. This phase separation prevents participation of bioactive substance in the chemical crosslinking reaction such as reaction between NHS ester and amine group. The separate phase also helps to modulate the release kinetics of active agent from the crosslinked material or gel, where ‘separate phase’ could be oil (oil-in water emulsion), biodegradable vehicle, and the like. Biodegradable vehicles in which the active agent may be present include: encapsulation vehicles, such as microparticles, microspheres, microbeads, micropellets, and the like, where the active agent is encapsulated in a bioerodable or biodegradable polymers such as polymers and copolymers of: poly(anhydride), poly(hydroxy acid)s, poly(lactone)s, poly(trimethylene carbonate), poly(glycolic acid), poly(lactic acid), poly(glycolic acid)-co-poly(glycolic acid), poly(orthocarbonate), poly(caprolactone), crosslinked biodegradable hydrogel networks like fibrin glue or fibrin sealant, caging and entrapping molecules, like cyclodextrin, molecular sieves and the like. Microspheres made from polymers and copolymers of poly(lactone)s and poly(hydroxy acid) are particularly preferred as biodegradable encapsulation vehicles.

In using crosslinked materials which are described herein as drug delivery vehicles, the active agent or encapsulated active agent may be present in solution or suspended form in crosslinker component or functional polymer solution component. The nucleophilic component, whether it be in the crosslinker or the functional polymer is the preferred vehicle due to absence of reactive groups. The functional polymer along with bioactive agent, with or without encapsulating vehicle, is administered to the host along with equivalent amount of crosslinker and aqueous buffers. The chemical reaction between crosslinker and the functional polymer solution readily takes place to form a crosslinked gel and acts as a depot for release of the active agent to the host. Such methods of drug delivery find use in both systemic and local administration of an active agent.

In using the crosslinked composition for drug delivery as mentioned above, the amount of crosslinkable polymer, crosslinker and the dosage agent introduced in the host will necessarily depend upon the particular drug and the condition to be treated. Administration may be by any convenient means such as syringe, cannula, trocar, catheter and the like.

Several methods for the formation of regional adhesion barriers are described, in which any of a variety of water soluble macromeric precursors are used. Preferably the functionality of a macromer molecule is >2 so that a crosslinked network or hydrogel results upon crosslinking.

In one embodiment, a crosslinked regional barrier is formed in situ, for example, by electrophilic-nucleophilic reaction, free radical polymerization initiated by a redox system or thermal initiation, wherein two components of an initiating system are simultaneously, sequentially or separately instilled in a body cavity to obtain widespread dispersal and coating of all or most visceral organs within that cavity prior to gelation and crosslinking of the regional barrier. Once the barrier is formed, the organs remain isolated from each other for a predetermined period, depending upon the absorption profile of the adhesion barrier material.

Preferably, the barrier is selected to have a low stress at break in tension or torsion, so as to not adversely affect normal physiological function of visceral organs within the region of application. The barrier also may contain a drug or other therapeutic agent.

Certain embodiments of the invention are accomplished by providing compositions and methods to control the release of relatively low molecular weight therapeutic species using hydrogels. In accordance with the principles of the present invention, a therapeutic species first is dispersed or dissolved within one or more relatively hydrophobic rate modifying agents to form a mixture. The mixture may be formed into microparticles, which are then entrapped within a bioabsorbable hydrogel matrix so as to release the water soluble therapeutic agents in a controlled fashion. Alternatively, the microparticles may be formed in situ during crosslinking of the hydrogel.

In one method of the present invention, hydrogel microspheres are formed from polymerizable macromers or monomers by dispersion of a polymerizable phase in a second immiscible phase, wherein the polymerizable phase contains at least one component required to initiate polymerization that leads to crosslinking and the immiscible bulk phase contains another component required to initiate crosslinking, along with a phase transfer agent. Pre-formed microparticles containing the water soluble therapeutic agent may be dispersed in the polymerizable phase, or formed in situ, to form an emulsion. Polymerization and crosslinking of the emulsion and the immiscible phase is initiated in a controlled fashion after dispersal of the polymerizable phase into appropriately sized microspheres, thus entrapping the microparticles in the hydrogel microspheres. Visualization agents may be included, for instance, in the microspheres, microparticles, and/or microdroplets.

Embodiments of the invention include compositions and methods for forming composite hydrogel-based matrices and microspheres having entrapped therapeutic compounds. In one embodiment, a bioactive agent is entrapped in microparticles having a hydrophobic nature (herein called “hydrophobic microdomains”), to retard leakage of the entrapped agent. More preferably, the composite materials that have two phase dispersions, where both phases are absorbable, but are not miscible. For example, the continuous phase may be a hydrophilic network (such as a hydrogel, which may or may not be crosslinked) while the dispersed phase may be hydrophobic (such as an oil, fat, fatty acid, wax, fluorocarbon, or other synthetic or natural water immiscible phase, generically referred to herein as an “oil” or “hydrophobic” phase).

The oil phase entraps the drug and provides a barrier to release by slow partitioning of the drug into the hydrogel. The hydrogel phase in turn protects the oil from digestion by enzymes, such as lipases, and from dissolution by naturally occurring lipids and surfactants. The latter are expected to have only limited penetration into the hydrogel, for example, due to hydrophobicity, molecular weight, conformation, diffusion resistance, etc. In the case of a hydrophobic drug which has limited solubility in the hydrogel matrix, the particulate form of the drug may also serve as the release rate modifying agent.

Hydrophobic microdomains, by themselves, may be degraded or quickly cleared when administered in vivo, making it difficult to achieve prolonged release directly using microdroplets or microparticles containing the entrapped agent in vivo. In accordance with the present invention, however, the hydrophobic microdomains are sequestered in a gel matrix. The gel matrix protects the hydrophobic microdomains from rapid clearance, but does not impair the ability of the microdroplets or microparticles to release their contents slowly. Visualization agents may be included, for instance, in the gel matrix or the microdomains.

In one embodiment, a microemulsion of a hydrophobic phase and an aqueous solution of a water soluble molecular compound, such as a protein, peptide or other water soluble chemical is prepared. The emulsion is of the “water-in-oil” type (with oil as the continuous phase) as opposed to an “oil-in-water” system (where water is the continuous phase). Other aspects of drug delivery are found in U.S. Pat. Nos. 6,632,457, 6,566,406, 6,703,047, 6,179,862, and 6,165,201, each of which are hereby incorporated by reference.

In another aspect of the present invention, the hydrogel microspheres are formed having a size that will provide selective deposition of the microspheres, or may linked with ligands that target specific regions or otherwise affect deposition of the microspheres within a patient's body.

Controlled rates of drug delivery also may be obtained with the system of the present invention by degradable, covalent attachment of the bioactive molecules to the crosslinked hydrogel network. The nature of the covalent attachment can be controlled to enable control of the release rate from hours to weeks or longer. By using a composite made from linkages with a range of hydrolysis times, a controlled release profile may be extended for longer durations.

Composite Biomaterials

The biocompatible crosslinked polymers of this invention optionally may be reinforced with flexible or rigid fibers, fiber mesh, fiber cloth and the like. The insertion of fibers improves mechanical properties like flexibility, strength, and tear resistance. In implantable medical applications, biodegradable fibers, cloth, or sheets made from oxidized cellulose or poly(hydroxy acid)s polymers like polylactic acid or polyglycolic acid, are preferred. Such reinforced structures may be produced using any convenient protocol known in the art.

In a preferred method, aqueous solutions of functional polymers and crosslinkers are mixed in appropriate buffers and proportions are added to a fiber cloth or net such as Interceed (Ethicon Inc., New Brunswick, N.J.). The liquid mixture flows into the interstices of the cloth and becomes crosslinked to produce a composite hydrogel. Care is taken to ensure that the fibers or fiber mesh are buried completely inside the crosslinked hydrogel material. The composite structure can be washed to remove side products such as N-hydroxysuccinimide. The fibers used are preferably hydrophilic in nature to ensure complete wetting of the fibers by the aqueous gelling composition.

Select Embodiments

An embodiment is a method of preventing adhesion in a uterus, the method comprising introducing a flowable material into a uterus to tamponade a surface of the uterus. The tamponade may be effective to reduce bleeding. The material may be a hydrogel. The material may be a stent. The material may separate at least two opposing portions of the surface to prevent contact between the two opposing portions. The material may substantially fill the uterus. The material may comprise a hydrophilic polymer. The material may comprise a polymer comprising the group —(CH₂CH₂O)—. The material may further comprise a therapeutic agent. The material may be degradable in vivo. The material may be hydrolytically degradable. The material may be degradable in vivo in less than about 7 days. The material may contact the surface for at least about one day. The material may be degradable in vivo in more than about one half day and in less than about 7 days. The material may be substantially formed in the uterus. The material may be partially formed outside the uterus and formation of the hydrogel may be completed in the uterus. The material may be formed from at least two chemically distinct precursors that react with each other to form the hydrogel. The at least two precursors may comprise a first precursor having a first functional group and a second precursor having a second functional group, wherein the first functional group reacts with the second functional group to form a covalent bond. The first functional group may comprise an electrophile and the second functional group may comprise a nucleophile. The electrophile may comprise a succinimide ester. The nucleophile may comprise an amine. The first functional group may comprise an amine. The first functional group may comprise a thiol. The method of claim 16 wherein the first precursor comprises at least three of the first functional group, or at least two, four six, or eight. The second precursor may comprises at least four of the second functional group or at least two, six, or eight. The material may be formed from at least one precursor that forms the hydrogel upon exposure to an activation agent. The at least one precursor may comprise a polymerizable functional group that comprises at least one vinyl moiety prior to exposure to the activation agent. The polymerizable functional group that comprises the at least one vinyl moiety may be, e.g., acrylate, methacrylate, methylmethacrylate. The polymerizable functional group may be polymerizable using free radical polymerization, anionic polymerization, cationic vinyl polymerization, addition polymerization, step growth polymerization, or condensation polymerization. The activation agent may be a polymerization initiator. The material may be formed by at least two polymers with opposite ionic charges that react with each other, a composition of a polymer comprising poly(alkylene) oxide and another polymer that undergoes an association reaction with the polymer comprising poly(alkylene) oxide, a thixotropic polymer that forms the hydrogel after introduction into the uterus, a polymer that from the hydrogel upon cooling, a polymer that forms physical crosslinks in response to a divalent cation, and a thermoreversible polymer. The material may comprise a natural polymer. The material may further comprise a visualization agent. An embodiment is a method of preventing adhesion in a uterus, the method comprising crosslinking at least one precursor to form a hydrogel in a uterus to tamponade a surface of the uterus. The hydrogel may be effective to reduce bleeding. At least one precursor may be dry.

### EXAMPLE

This Example demonstrates the easiness of use, safety, and effectiveness of the hydrogel barrier SPRAYGEL, provided by Confluent Surgical, Boston, Mass., and used herein as an intrauterine adhesion barrier. Portions of this Example were submitted for publication to The American Association of Gynecologic Laparoscopists for its 2004 annual meeting with the title Initial feasibility study of an hydrogel adhesion barrier system in patients treated by operative hysteroscopy for intrauterine benign pathologies.

In brief, twenty consecutive patients undergoing operative hysteroscopy were enrolled. Patients were being treated for, e.g., endometrial polyps, submucosal myomas, sinechiae, or uterine Mullerian anomalies (septa). Patients with malignancies, pregnancies, or lesions not suitable for hysteroscopic treatment were excluded. Each patient was evaluated preoperatively by a transabdominal/transvaginal ultrasound, a pregnancy test and a diagnostic hysteroscopy with or without a biopsy. After surgery, the patient's uterine cavity was filled with SPRAYGEL and the patients were all evaluated postoperatively by ultrasound after 7, 14, 21 days and by hysteroscopy after 1 and 2 months. SPRAYGEL showed facility of use with a mean application time of 1.14 min. (max 2 min.). The mean amount of hydrogel requested to fill the cavity was 6.14 ml. (max 10 ml.) depending from uterine cavity size. SPRAYGEL was completely reabsorbed within 21 days (min. 14 days) in all cases and not found at the first postoperative hysteroscopy in any case. No adverse effect, complications, or postoperative intrauterine adhesions were observed.

SPRAYGEL and the dual syringe application catheters were provided by Confluent Surgical (Waltham, Mass.). Twenty consecutive patients with diagnosis of abnormal menstrual bleeding were treated with the hydrogel. Table 1. The hysteroscopic diagnostic procedures were ambulatory, performed using a rigid hysteroscope (Wolf, Germany) while operative procedures were performed in the surgical room using a 27 Fr scope (Wolf, Germany). Upon completion of the surgical procedures, resections for polyps and submucosal myomas and ablations for endometrial benign hyperplasia, the uterine cavity was drained of distension fluid (mannitol-sorbitol mix) with a 200 ml. of saline solution irrigation. Between 4 and 10 ml of SPRAYGEL were applied in the uterine cavity by advancing the 8 Fr applicator to the fundus. Paracervical block was administered as anesthesia in every procedure. No post-operative oestrogen replacement therapy was given to the patients after the initial hysteroscopy. Patients were evaluated postoperatively by ultrasound after 7, 14, and 21 days and by hysteroscopy after 1-2 months.

TABLE 3
Patient Demographics

| | SPRAYGEL Treated |
|---|---|
| | Group |
| N | 20 |
| Mean Age | 42 |
| Range | Min. 31-Max. 61 |
| Surgical Procedure | |
| Polyposis (removal) | 8 |
| Hyperplasia (endometrial ablation) | 6 |
| Submucosal myomas (removal) | 6 |
| Septa | 0 |

The assessment of the efficacy of SPRAYGEL was based on the presence, extent and severity of intrauterine adhesions at the follow-up hysteroscopy performed after 1-2 months. Table 2 shows the scoring system used for the evaluations of postoperative adhesions:

TABLE 4
March et al., classification, 1978 of IUA by hysteroscopic findings

| Grade | Findings |
|---|---|
| Severe | More than three quarters of uterine cavity involved |
| | Agglutination of walls or thick bands |
| | Ostial areas or upper cavity occluded |
| Moderate | Between one quarter-three quarters of the uterine cavity |
| | involved |
| | No agglutination of walls, adhesions only |
| | Ostial areas and upper cavity only partially occluded |
| Minimal | Less than one quarter of uterine cavity involved: thin or filmy |
| | adhesions |
| | Fundus, ostial areas are clear |

All patients were asymptomatic and free of adhesions at time of the second look hysteroscopy. Thus, no grade was assigned. SPRAYGEL was found to be easy to use with a mean application time of 1.1 min. (max 2 min.). On average 6 ml of SPRAYGEL was found to be appropriate to fill the uterine cavity (max 10 ml.) SPRAYGEL was completely reabsorbed within 21 days (min. 14 days) in all cases and no residual hydrogel was found at the follow-up hysteroscopies. There were no adverse events, complications and incidence of postoperative pain.

TABLE 5
Results

| | Mean | Max | Min |
|---|---|---|---|
| Op. time (min.) | 17.6 | 33 | 10 |
| Application time | 1.1 | 2 | 1 |
| (min.) | | | |
| Amount of | 6.1 | 10 | 4 |
| SPRAYGEL | | | |
| required (ml.) | | | |
| Easiness of use (1- | 1.1 | 2 | 1 |
| 2-3) | | | |
| Time of | 17.5 | 21 | 14 |
| reabsorption | | | |
| Complications | 0 | 0 | 0 |
| Adverse effects | 0 | 0 | 0 |
| Adhesion | 0 | 0 | 0 |
| Incidence | | | |

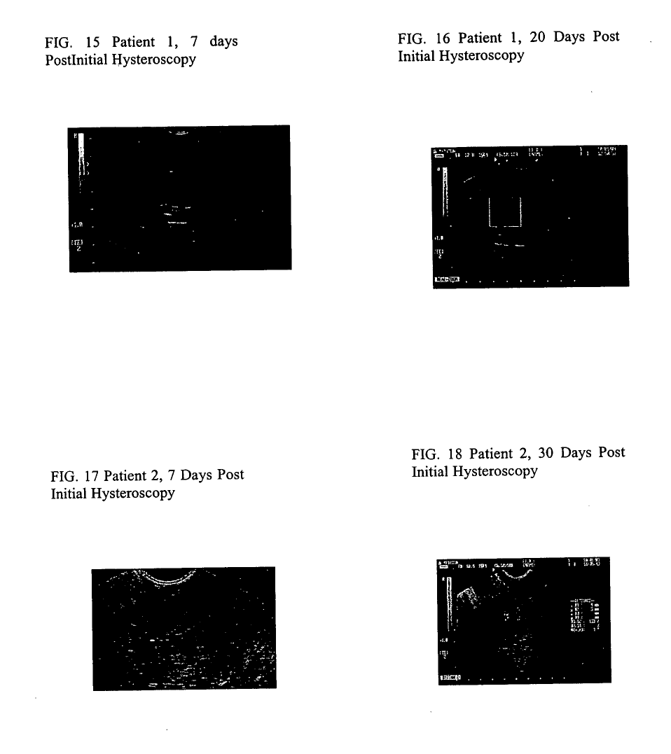

The absorption of SPRAYGEL was confirmed by ultrasound at 3-4 weeks as shown in FIGS. 15-18. At 7 days SPRAYGEL distended the uterine walls. The walls were found to be collapsed, but not adherent at the follow-up hysteroscopy.

Additionally, while not specifically recorded for this study, it appeared that the presence of SPRAYGEL within the uterus apparently served to tamponade post-surgical bleeding. At follow up hysteroscopy, about 2 ml of a yellowish fluid was noted to be residual within the uterus, this was presumed to be traces of blood and fibrin that had been tamponaded by SPRAYGEL post-surgery.

Since no adhesions were noted at follow-up hysteroscopies, it was determined that SPRAYGEL does not create agglutination of injured adjacent surfaces and appears to be promising as a barrier to prevent intrauterine adhesion formation. SPRAYGEL appears to be well tolerated when administered within the uterine cavity after hysteroscopic surgery. Complete absorption of the hydrogel was verified by ultrasound and second look hysteroscopy. Patients appeared to tolerate the hydrogel well and did not report any pain due to the presence of this device within the uterine cavity. Since the hydrogel is absorbable, it presents advantages over other adhesion prophylaxis approaches, such as placement of balloon catheters that need subsequent removal.

### REFERENCES

¹ Diamond, M. P. et al.: Adhesion Reformation and De Novo Adhesion formation After Reproductive Pelvic Surgery. Fert. & Ster. 47:5, 1987
² Diamond, M. P. et al.: Pathogenesis of Adhesion Formation/reformation: Application to Reproductive Pelvic Surgery. Microsurg. 8:103-107, 1987
³ Gomel, V. et al.: Pathophysiology of Adhesion Formation and Strategies for Prevention. J. Repro. Med. 41:1, 1996
⁴ dizerega, G. S.: Use of Adhesion Prevention Barriers in Ovarian Surgery, Tubalplasty, Ectopic Pregnancy, Endometriosis, Adhesiolysis, and Myomectomy. Curr. Opin. Obstet. Gynechol. 8:3, 1996
⁵ Mettler L, Audebert A, Lehmann-Willenbrock E, Schive K, Jacobs V R: A Prospective Clinical Trial of SPRAYGEL as a Barrier to Adhesion Formation: Interim Analysis. J Am Assoc Gynecol Laparosc 10 (3):339-344, 2003.
⁶ Johns D A, Ferland R, Dunn R,: Initial feasibility study of a sprayable hydrogel adhesion barrier system in patients undergoing laparoscopic ovarian surgery. J Am Assoc Gynecol Laparosc 10 (3):334-338, 2003.
⁷ Zhou, X. and Harris, M. J., Novel Degradable Poly(Ethylene glycol) Esters for Drug Delivery, PEG Chem. Biol. Appl., ACS Symp. Ser. 680, 458-72, 1997.
⁸ The Polyglycol Handbook, Dow Chemical Co.
⁹ Drug Facts and Comparisons. Facts and Comparisons, Publishers, St. Louis Mo. 1996.
¹⁰ March C, Israel R, March A: Hysteroscopic management of intrauterine adhesions. Am J Obstet Gynecol 1978; 130: 653
¹¹ Raziel A., Arieli Sholmo: Investigation of the uterine cavity in recurrent aborters. Fertil Steril 1994; 62: 5, 1080-1082.
¹² Hesham Al-Inany. Intrauterine adhesions. An update. Acta Obstet Gynecol Scand 2001; 80: 986-993.
Also: Schenker J G, Margalioth E J. Intrauterine adhesions. An updated appraisal. Fertil Steril 1982; 37: 593-610.

All references, publications, patents, and patent applications set forth in this application are hereby incorporated by reference herein.

1. A method of preventing adhesion in a uterus, the method comprising introducing a flowable material into a uterus to tamponade a surface of the uterus.

2. The method of claim 1, wherein the tamponade is effective to reduce bleeding.

3. The method of claim 1, wherein the material is a hydrogel.

4. The method of claim 1, wherein the material acts as a stent to keep the uterine walls apart.

5. The method of claim 1, wherein the material separates at least two opposing portions of the surface to prevent contact between the two opposing portions.

6. The method of claim 1, wherein the material substantially fills the uterus.

7. The method of claim 1, wherein the material comprises a hydrophilic polymer.

8. The method of claim 1, wherein the material comprises a polymer comprising the group —(CH₂CH₂O)—.

9. The method of claim 1, wherein the material further comprises a therapeutic agent.

10. The method of claim 1, wherein the material is degradable in vivo.

11. The method of claim 10, wherein the material is hydrolytically degradable.

12. The method of claim 10, wherein the material is degradable in vivo in less than about 7 days.

13. The method of claim 10, wherein the material contacts the surface for at least about one day.

14. The method of claim 10, wherein the material is degradable in vivo in more than about one half day and in less than about 7 days.

15. The method of claim 1, wherein the material is substantially formed in the uterus.

16. The method of claim 1, wherein the material is partially formed outside the uterus and formation of the hydrogel is completed in the uterus.

17. The method of claim 1, wherein the material is formed from at least two chemically distinct precursors that react with each other to form the hydrogel.

18. The method of claim 17, wherein the at least two precursors comprise a first precursor having a first functional group and a second precursor having a second functional group, wherein the first functional group reacts with the second functional group to form a covalent bond.

19. The method of claim 18, wherein the first functional group comprises an electrophile and the second functional group comprises a nucleophile.

20. The method of claim 19, wherein the electrophile comprises a succinimide ester.

21. The method of claim 19, wherein the nucleophile comprises an amine.

22. The method of claim 18, wherein the first functional group comprises an amine.

23. The method of claim 18, wherein the first functional group comprises a thiol.

24. The method of claim 18, wherein the first functional group comprises a member of the group consisting of imines, carboxyls, isocyanates, carbodiimidazole, sulfonyl chloride, chlorocarbonates, n-hydroxysuccinimidyl ester, succinimidyl ester, sulfasuccinimidyl esters, aryl halides, sulfosuccinimidyl esters, N-hydroxysuccinimidyl esters, succinimidyl esters, epoxides, aldehydes, maleimides, and imidoesters.

25. The method of claim 18, wherein the first precursor comprises at least three of the first functional group.

26. The method of claim 18, wherein the second precursor comprises at least four of the second functional group.

27. The method of claim 1, wherein the material is formed from at least one precursor that forms the hydrogel upon exposure to an activation agent.

28. The method of claim 27, wherein the at least one precursor comprises a polymerizable functional group that comprises at least one vinyl moiety prior to exposure to the activation agent.

29. The method of claim 27, wherein the polymerizable functional group that comprises the at least one vinyl moiety is acrylate, methacrylate, or methylmethacrylate.

30. The method of claim 27, wherein the polymerizable functional group is polymerizable using free radical polymerization, anionic polymerization, cationic vinyl polymerization, addition polymerization, step growth polymerization, or condensation polymerization.

31. The method of claim 24, wherein the activation agent is a polymerization initiator.

32. The method of claim 1, wherein the material is formed by at least two polymers with opposite ionic charges that react with each other, a composition of a polymer comprising poly(alkylene) oxide and another polymer that undergoes an association reaction with the polymer comprising poly(alkylene) oxide, a thixotropic polymer that forms the hydrogel after introduction into the uterus, a polymer that from the hydrogel upon cooling, a polymer that forms physical crosslinks in response to a divalent cation, and a thermoreversible polymer.

33. The method of claim 1, wherein the material comprises a natural polymer.

34. The method of claim 1, wherein the material further comprises a visualization agent.

35. The method of claim 1, wherein the material further comprises an imaging agent.

36. The method of claim 35, wherein the imaging agent can be imaged by X-ray or ultrasound.

37. A method of preventing adhesion in a uterus, the method comprising crosslinking at least one precursor to form a hydrogel in a uterus to tamponade a surface of the uterus.

38. The method of claim 37, wherein the hydrogel is effective to reduce bleeding.

39. The method of claim 37, wherein the at least one precursor is dry.

40. The method of claim 37, wherein the crosslinking is covalent crosslinking.

41. The method of claim 37, wherein the hydrogel acts as a stent.

42. The method of claim 37, wherein the hydrogel separates at least two opposing portions of the surface to prevent contact between the two opposing portions.

43. The method of claim 37, wherein the hydrogel substantially fills the uterus.

44. The method of claim 37, wherein the hydrogel comprises a hydrophilic polymer.

45. The method of claim 37, wherein the hydrogel comprises a polymer comprising the group —(CH₂CH₂O)—.

46. The method of claim 37, wherein the hydrogel further comprises a therapeutic agent.

47. The method of claim 37, wherein the hydrogel is degradable in vivo.

48. The method of claim 47, wherein the hydrogel is hydrolytically degradable.

49. The method of claim 47, wherein the hydrogel is degradable in vivo in less than about 14 days.

50. The method of claim 47, wherein the hydrogel contacts the surface for at least about one day.

51. The method of claim 47, wherein the hydrogel is degradable in vivo in more than about one half day and in less than about 14 days.

52. The method of claim 37, wherein the hydrogel is substantially formed in the uterus.

53. The method of claim 37, wherein the hydrogel is partially formed outside the uterus and formation of the hydrogel is completed in the uterus.

54. The method of claim 37, wherein the hydrogel is formed from at least two chemically distinct precursors that react with each other to form the hydrogel.

55. The method of claim 54, wherein the at least two precursors comprise a first precursor having a first functional group and a second precursor having a second functional group, wherein the first functional group reacts with the second functional group to form a covalent bond.

56. The method of claim 55, wherein the first functional group comprises an electrophile and the second functional group comprises a nucleophile.

57. The method of claim 56, wherein the electrophile comprises a succinimide ester.

58. The method of claim 56, wherein the nucleophile comprises an amine.

59. The method of claim 55, wherein the first functional group comprises an amine.

60. The method of claim 55, wherein the first functional group comprises a thiol.

61. The method of claim 55, wherein the first functional group comprises a member of the group consisting of imines, carboxyls, isocyanates, carbodiimidazole, sulfonyl chloride, chlorocarbonates, n-hydroxysuccinimidyl ester, succinimidyl ester, sulfasuccinimidyl esters, aryl halides, sulfosuccinimidyl esters, N-hydroxysuccinimidyl esters, succinimidyl esters, epoxides, aldehydes, maleimides, and imidoesters.

62. The method of claim 55, wherein the first precursor comprises at least three of the first functional group.

63. The method of claim 55, wherein the second precursor comprises at least four of the second functional group.

64. The method of claim 37, wherein the hydrogel is formed from at least one precursor that forms the hydrogel upon exposure to an activating agent.

65. The method of claim 37, wherein the at least one precursor comprises a polymerizable functional group that comprises at least one vinyl moiety prior to exposure to the activating agent.

66. The method of claim 65, wherein the polymerizable functional group that comprises the at least one vinyl moiety is acrylate, methacrylate, or methylmethacrylate.

67. The method of claim 65, wherein the polymerizable functional group is polymerizable using free radical polymerization, anionic polymerization, cationic vinyl polymerization, addition polymerization, step growth polymerization, or condensation polymerization.

68. The method of claim 65, wherein the activating agent is a polymerization initiator.

69. The method of claim 37, wherein the hydrogel is formed by at least two polymers with opposite ionic charges that react with each other, a composition of a polymer comprising poly(alkylene) oxide and another polymer that undergoes an association reaction with the polymer comprising poly(alkylene) oxide, a thixotropic polymer that forms the hydrogel after introduction into the uterus, a polymer that from the hydrogel upon cooling, a polymer that forms physical crosslinks in response to a divalent cation, and a thermoreversible polymer.

70. The method of claim 37, wherein the hydrogel comprises a natural polymer.

71. The method of claim 37, wherein the hydrogel further comprises a visualization agent.

72. The method of claim 37, wherein the hydrogel further comprises an imaging agent.

73. The method of claim 71, wherein the imaging agent is for imaging by X-ray or ultrasound.

74. A method of treating a uterus, the method comprising introducing a precursor into a uterus to form a material comprising the precursors in situ in the uterus that contacts a tissue in the uterus.

75. The method of claim 74, wherein the material is a hydrogel.

76. The method of claim 74, wherein the material separates at least two opposing portions of the tissue to prevent contact between the two opposing portions.

77. The method of claim 74, wherein the material substantially fills the uterus.

78. The method of claim 74, wherein the material comprises a hydrophilic polymer.

79. The method of claim 74, wherein the material is degradable in vivo.

80. The method of claim 79, wherein the material is degradable in vivo in less than about 14 days.

81. The method of claim 74, wherein the material is partially formed outside the uterus and formation of the hydrogel is completed in the uterus.

82. The method of claim 74, wherein the material is formed from at least two chemically distinct precursors that react with each other to form the hydrogel.

83. The method of claim 82, wherein the at least two precursors comprise a first precursor having a first functional group and a second precursor having a second functional group, wherein the first functional group reacts with the second functional group to form a covalent bond.

84. The method of claim 74, wherein the material is formed from at least one precursor that forms the hydrogel upon exposure to an activation agent.

85. The method of claim 74, wherein the material further comprises a visualization agent.

86. The method of claim 74, wherein the material further comprises an imaging agent.
